(19) United States
(12) Patent Application Publication
(10) Pub. No.: US 2023/0324064 A1
(43) Pub. Date: Oct. 12, 2023

(54) Air Quality Turnover Solution System, Device and Methods to Mitigate the Risk of Infection in Room Turnover

(71) Applicants: Marc Garbey, Houston, TX (US); Shannon Furr, Pearland, TX (US); Guillaume Joerger, Strasbourg (FR)

(72) Inventors: Marc Garbey, Houston, TX (US); Shannon Furr, Pearland, TX (US); Guillaume Joerger, Strasbourg (FR)

(21) Appl. No.: 18/042,211

(22) PCT Filed: Aug. 18, 2021
(86) PCT No.: PCT/US21/46571

§ 371 Date: Feb. 18, 2023

(51) Int. Cl. F24F 11/39; A61G 10/02; F24F 11/49; G05B 19/042

(52) U.S. Cl. CPC F24F 11/39; A61G 10/02; F24F 11/49; G05B 19/042; F24F 2110/64

(57) ABSTRACT

The present invention relates to a means of reducing exposure to aerosolized particulate pathogens thereby (1) safely increasing throughput, (2) increasing surgical flow rates and (3) improving surgical suite turnover times (i.e., limiting non-productive facility space utilization) through air quality monitoring, generally. Further, the present invention couples air quality sensors with real-time processing technology by combining algorithmic enabled and mechanical means of lowering risk levels of both patients, medical professionals and ancillary staff alike.

### CROSS REFERENCE TO RELATED APPLICATIONS

The present application claims priority to previously filed U.S. Provisional Patent Application Ser. Nos. 63/067,036 and 63/119,952 filed Aug. 18, 2020, and Dec. 1, 2020, respectfully, the entirety of each which are incorporated by reference herewith.

### FIELD OF THE INVENTION

The present invention relates to a system, employed apparatuses and method of (1) reducing exposure to aerosolized particulate pathogens thereby (2) safely increasing throughput, (3) increasing surgical flow rates and (4) improving surgical suite turnover times (i.e., limiting non-productive facility space utilization) through air quality monitoring. Further, the present invention couples air quality analysis and monitoring with real-time detection and processing technology thereby combining a means of detecting, analyzing and reporting surgical suite air quality thereby lowering exposure risk levels of patients, medical professionals and ancillary staff alike.

### BACKGROUND

January 2020, the World Health Organization (WHO) issued a global health alert for a novel coronavirus named severe acute respiratory syndrome coronavirus 2 (SARS-CoV-2) that caused an acute respiratory infection disease (COVID-19) which originated in Wuhan, Hubei Province, China. The COVID-19 pandemic, as declared by the WHO on March 2020, has led to over 200 million confirmed cases (as of Aug. 5, 2021), including over 4 million worldwide deaths (as of Jul. 7, 2021) as reported to the WHO.¹ The role of ambient and indoor particulate matter as potential carriers for SARS-CoV-2 was quickly under investigation. Aerosol and droplets generated during speaking, or coughing are well-known as the source of transmission pathways for all kind of viral and bacterial infections. In general, ambient air particulate matter contain various chemical and biological constituents (bacteria, virus or fungi) of great health concern. This is even more critical in a hospital setting where patients are very vulnerable. An event such as the pandemic of SARS-CoV-2, as detailed below, underlines the necessity of hospital occupants' safety at all time and air quality is on top of the monitoring list. Ventilation system are rarely the source of pathogens but can become a reservoir that can quickly create exogenous infections throughout the facility. Surgical theaters are not spared and present even more complex environment with positive and negative pressure, staff coming in and out, and can become a source of dangerous particulate matter during specific surgeries.

The need among systematic SARS-CoV-2 cases for hospitalization, and use of finite hospital resources, has shifted surgical requirements away from both elective and non-elective surgeries, wherein hospitals have intensely reduced all surgical activities, including acute surgeries, in favor of addressing increasingly high numbers of COVID admissions, primarily from unvaccinated individuals. Yet, the need for essential surgeries continues unabated and related safety requirements and protocols for elective and non-elective surgeries alike have only intensified as the current pandemic continues. Markedly, in a multitude of cases, far-reaching strenuous effects on the US healthcare system remain and, in most cases, intensifies.

Given the above, there is little doubt that the present pandemic will reverberate through the surgical practice of medicine ranging from workforce distribution, staffing realignment, procedural prioritization, evaluation of current pathogenic transmission risk models intraoperatively as well as impact surgical education (training and retraining) influencing the entire state of Healthcare for years and decades to come.

And while long-terms effects cannot be adequately evaluated currently, short term effects have an immediacy which is manifest. These effects include cancellations, rescheduling, postponements (elective, non-urgent and critical procedures), augmented visitation schedules, changes in pre and postoperative care, altered patient education, reassignment and redistribution of staff, modified surgical techniques and reprioritized urgency triage. Further evidenced are repurposing of ORs and surgical suites for critical care, constraints on tight resources and budgeting, risk-balanced consideration of non-surgical alternatives, telemedicine for non-contact consultations, modified surgical pathways within facilities, operating room workflow reconsiderations, surgical flowthrough analysis, open versus minimally invasive techniques (and each's merits) as well as COVID-19 positive patient care and designated areas.^{2,3}

Chief among these, though, may be the postponement of essential surgical procedures due to the secondary and tertiary COVID-19 effects (e.g., limited and repurposed staff, occupied spaces, conversions of surgical suites to SARS-CoV-2 patient areas), all inevitably leading to worsening conditions, sicker patients and more advanced disease progression wherein surgeries, when performed, are more complex, lengthier and more consuming of restricted resources. Too, showing no sign of abatement, is the average of roughly 19 million upper and lower endoscopy procedures which were completed (per year, prior to COVID-19) in the US alone which, if pushed out, will lead to disease progressions and a potential bottle neck of GI procedures well into the foreseeable future.^{4,5}

In terms of surgery staff, while the risk of acute respiratory infection (ARI) transmission through surgical care services is not completely defined, it is evident that healthcare workers have a significantly increased risk of contracting the SARS-CoV-2 simply in terms of contact and proximity. In fact, this close juxtaposition of staff to patients gives way to additional and often more stringent infection control measures for surgeons providing services on SARS-CoV-2 positive patients as well as those patients in which SARS-CoV-2 status is asymptomatic, pre-symptomatic, false negative and/or presumed positive. In fact, GI surgical patients, as a discrete patient population, present as a high-risk group for health care workers where those patients are often at increased ages (with less robust immune systems), warranting heightened surgical care services, wherein patients in this category also inherently demand close physical contact, provide small droplet fluid exposure to staff (via endemic aerosol generating procedures) all contributing to a high potential for ARI (Acute Respiratory Infection) transmission. Moreover, recent data points to infection transmission even between and among those who are vaccinated⁶ as well as those patients who are pre-symptomatic⁷ and/or positive/asymptomatic.^{8,9} This increased exposure risk due to proximity, combined with risks inherent with intimate surgical relationship and patient population, justifies treating all patients, positive or negative, as presumed positive for all respiratory transmitted disease.

And while the present application is drawn to mitigation and reduction of the transfer of COVID-19, including the current pandemic and variants of SARS-CoV-2, it stands as an exemplary transmission model providing a relational and representative example via its applicability to aerosolized antigens, in general. Therefore, inasmuch as SARS-CoV-2 is universal in its application to all airborne particulates transmitted through respiratory droplets, knowing the capacity for harboring antigens may be extrapolated to ranges below 2.5 microns, above 2.5 microns and otherwise between and among all pathogens.¹⁰

While strides have been made to overcome the inadequacies of maintaining appropriate air quality in a surgical area, it remains evident that considerable failings in terms of risk mitigation and adequate air ventilation remain in the field. It is the goal of the present invention to remedy these shortcomings as to allow more efficient monitoring and management of the air quality in “real-time” of surgical spaces and to offer a system of improved management of the physical and biological actions of small particles within a confined surgical space to more accurately predict small molecule dynamics and potential antigenic virulence. It is a stated goal by inventors to mitigate relative pathogenicity within a surgical space by confirming and assuring actual adequate air quality (as opposed to theoretical prediction based on the 99% elimination threshold given by HVAC system model formulas) thereby providing a quantitative measure of confirming an acceptable level of air particles (especially those under 5 microns in diameter) and lessening the calculatable time between procedures accurately within said space. Added, the present invention and its ability to protect at-risk individuals may be extrapolated to other non-surgical applications.

### SUMMARY

The primary route of transmission of SARS-CoV-2 as well as other aerosolized respiratory viruses is through airborne transmission and inhalation of infectious aerosolized droplets.¹¹ Historically this conveyance has focused on droplets 5 μm and above,¹² yet greater attention and focus has been directed explicitly to those particles 5 μm (microns) and less. Unequivocally, large respiratory droplets (those >5 μm in diameter) are involved in short-range transmission while droplets <5 μm (specifically 2.5 microns or less) are responsible for transmission over greater spatial distances. What is more, those respiratory droplets ranging in the 2.5 microns in diameter or less, tend to stay aloft for extended periods of time (up to and including indefinite periods) and may reach distances of up to 2 meters¹³ where those small particles share an inverse relationship of depth of penetration to particle size and larger droplets penetrating a depth shallower in the respiratory tract compared to smaller droplets (travelling further distances into the bronchioles and alveoli).¹⁴

The SARS-CoV-2 virion (i.e., the complete, infective form of a virus outside a host cell, including a core of RNA or DNA with accompanying capsid) is on the order of 50 to 200 nanometers in diameter. For reference, most viruses range from 20 nm in diameter (Parvoviridae Picornaviridae and Circovirus) to between 250 to 400 nm in diameter (Mimi virus in some cases exceeding 400 nm). For perspective, HIV can exist in the 120 nm range while zika virus is considerably smaller at 45 nanometers. In comparison, a dust mite exists in the 200 μm range and respiratory droplets themselves can range in the 1 to 10 micrometers range (i.e., micrometers being 1000 times the size of a nanometer) wherein even the smallest detectable particle respiratory droplets (e.g., 2.5 microns or less) express a verifiable probability that a droplet contains one or more virions—which scales with its initial hydrated volume. That is, as the cube of its diameter, d, which, with an oral fluid average virus RNA load of 7×106 copies per milliliter (maximum of 2.35-109 copies per milliliter), the probability that a 10-μm-diameter droplet, prior to any dehydration, contains at least one virion is ˜0.37% and the probability for a 1 μm-diameter being 0.037%.¹⁵ Moreover, as diameter decreases, due to evaporation, terminal velocity of a falling droplet scales as the square of its diameter and the decreasing size of droplets only potentiating a slowing descent.

It is therefore imperative to understand the applicability of aerosolization and the mechanisms of airborne transmissibility of SARS-CoV-2 virions, as well as various other potential viral vectors of disease transmission, in relation to each's ability to (1) reside and exist within said small particle (2.5 microns and less) aerosolized vehicles as well as (2) to effectively remain airborne and travel from an infected individual to a virus naïve host.

Patently, in terms of aerosol generating procedures (i.e., perioperative anesthesiological management, tracheal intubation, extubation, tracheotomy and manual ventilation before intubation), procedures are recognized as significantly associated with an increased risk of transmission not similarly associated with endotracheal aspiration, nasogastric tube insertion, nebulizer treatment and high flow O₂.¹⁶ Additionally, airway management-related aerosols, bodily fluids, surgical smoke (described in detail below), laser vapor, contaminated insufflation, and specimen handling run an equally appreciable risk of transmission.^{17,18,19,20}

It is therefore incumbent upon the healthcare community to understand (a) the primary MOA (mode of action) of airborne respiratory pathogen transmission (as stated above), (b) those areas which are amendable to intervention and transmission interference and (c) the means by which those interferences may be best effectuated.

Inventors advance a practical solution to mitigate patient and surgical staff risk to airborne infective agents and to improve the turnover process in a procedural room from both the perspective of safety and efficiency. Inventors herein provide examples coming from a workflow application in operating theaters and in gastroenterology clinics and suites treating a large number of patients with general surgery such as appendectomy or cholecystectomy and gastroenterology (GI) procedures such as colonoscopy, esophagogastroduodenoscopy (EGD), or Endoscopic Retrograde Cholangiopancreatography (ERCP) where surgical masks, designed for gross (large) particles (those above 5 μm), prove inadequate for smaller infectious particles and where respirators suffer from improper implementation which may pose a significant factor in infection transmission.

The medical staff that are present during the procedure are required to wear appropriate personal protective equipment (PPE) which is not, in and of itself, failproof (see above). Yet, current, and next, patients in a surgical suite may be particularly vulnerable and risking potential exposure as well, if the air contamination has not been adequately eliminated by the Heating, Ventilation and Air Conditioning (HVAC) system. Moreover, given a series of concomitant factors, this particular patient population is especially susceptible to potential infection transmittance including: (1) inability to wear a mask (due to anesthesia administration/nature of endoscopic procedures), (2) potential immune system suppression naturally a consequence of requiring a GI procedure (patients often exhibiting inflammation and malnutrition where sick patient often exhibit reflex and reflux producing sick patients), (3) proliferation of medical gas infiltrates due to the nature of procedures, (4) patient stress commonly induced by a surgical intervention, (5) the natural ‘gag’ reflex at the beginning of the procedure inducing cough generating aerosols, and (6) age and related immune system effects. Add to this the fact that small particles (those less than 2.5 microns in diameter), especially aerosolized small particles, constitute an especially insidious risk wherein small particles (e.g. typically most effectively conveyed by smaller airborne water droplets) (a) stay aloft for longer periods (as opposed to larger particles), (b) may penetrate mask materials, (c) depend on aerosolization (in addition to droplet-ladened fomites) for virulence, and (4) can penetrate further in the respiratory tree (e.g. alveoli) where the functional aspects of a viral affront depends on multiple factors (e.g. viral load, time of exposure, conditional health status and even individual susceptibility) that are manifest and amplified in the surgical setting.

Inventors proffer that they are first to advance and prove quantitatively that GI procedures generate significant level of aerosol having continuously monitored about 1500 procedures during 50 clinic days and count the Small Particle Count (SPC) generated by the procedure during the procedures using an off-the-shelf particle counter (sensors) based on current laser optical technology. Significantly, inventors have shown that the number of particles in the procedural room more than double during the first few minutes of the procedure, when a flexible endoscope is introduced in the patient, but particles may remain a loft for extended periods subject to room conditions, surgical equipment, medical staff and turbulent flow (among other variables).

To monitor and access air quality, inventors have designed a new cyber-physical system with two to a plurality of sensors (for disparate as well as redundant sampling) in each procedural room of low, medium and high-volume GI outpatient center where inventors conducted studies incorporating engineering developments providing a stable system that seeks to provide an uninterrupted connection, with integrated redundancy and signal protections, ensuring a very high small particle measurement accuracy and low probability of information loss.

The system as shown and described, consists of, at least the following:
1. A “computer vision sensor” that checks the endoscope's video view and automatically retrieves the state of the endoscope in real-time (e.g., off, read for use, in use). To this end, inventors instituted extensive procedures and conducted enumerable quantitative analyses in the development of an algorithm to generate robust and accurate measures that holds an accuracy of timing of one minute or less;
2. A Dylos® air quality sensor (or, alternatively, inventor designed device provided herein) on one wall (to a plurality of walls) of the procedural room where these procedural rooms are relatively small compared to surgery operating rooms and more amendable to fewer quality sensors. According to our Computational Fluid calculation CFD calculation ref [Garbey2020a],²³ air mixing takes less than a minute to give a relatively uniform SPC per the room's volume. Therefore, particle count has little time lag compared to the source of particles (albeit medical staff or the patient). Further, the location of the sensor is not position sensitive (i.e., is robust and accurate in function at several positions) unless it is segregated or occluded (in a room corner, behind surgical equipment, or the like, which serves to obstruct the airflow through the sensor); and
3. Various ancillary sensors (ex. gas sensors, temperature sensors, pressure sensors, light sensors, motion sensors) as necessity dictates.

Sensors provide digital information in quasi-real time (i.e., an appreciably short time period) which is then sent via wireless local area network (LAN) to a server that archives the time series into a database. Preferably, the server itself is implemented behind the institution's firewall, so all data and network communications are protected in the same manner as the institutions own data. Significantly, and in observation of various HIIPA and state and federal regulations, the data retrieved by inventors' system is designed to be patient agnostic, contain no patient identifiable or protected health information, where the flexible endoscopy video feed does not leave the endoscope tower nor is this information saved or archived onto inventors' system at any point.

As per the manufacturers specifications, the Dylos® laser particle counter, used extensively in the present experiments, is configured to test an average particle count every minute in a unit system with units denoted ud that correspond to 0.01 particles per cubic foot, or 0.00028 particles per cubic meter (1 cubic foot=0.0283168 cubic meter). According to Smart Air® [http://smartairfilters.com/cn/en/], the Dylos® system's output is highly correlated (r=0.8) to a “ground true” measurement provided by comparable high-end system, such as the Sibata LD 6S that is claimed to be accurate within 10% c in controlled laboratory conditions. According to SmartAir®, the Dylos® system displays particularly high accuracy at the lower concentration ends, which is of pointed interest for this study's purposes.²¹ also compared the Dylos® system with a more expensive system: the Sidepak AM510 Personal Aerosol Monitors (TSI, Minnesota, USA). Semple et al. concluded that the Dylos® system's output agrees closely with the one produced by the Sidepak instrument with a mean difference of 0.09 micro gram per cubic meter (mu g/m³). Alternative devices to provide SPC are proposed by inventors and are included herewith.

Inventor's further use additional sensor(s) tracking aerosol generated by the evaporation of alcohol product where this additional sensor gives the present system both the time of cleaning and duration the janitorial team is cleaning the procedural room after a surgical intervention. As is described, inventors may use one to a plurality of sensors to discriminate SPC coming from any sources (ex. aerosolized patient exudate) from that generated specifically by the chemical product used in cleaning the procedural room.

The present invention is the combination of a hardware and software solution with a mathematically based algorithm to compute, in real time before, during and after each procedure, the air quality turnover (i.e. how long the room needs to be closed in order to retrieve an “excellent” air quality) to protect the patient currently undergoing a procedure, mitigate the risk of airborne contamination for the next patient's procedure, to provide a means to deliver assurances to both patients and operational medical staff as to air quality/mitigated risk of infection, to protect essential staff and cleaning workers from potential contamination and to provide institutions quantitative data to confidently decrease times between procedure.

In principle once the room is cleaned, the doors of the procedural room must be closed to allow the HVAC system to renew the air and eliminate airborne particles to a significant and appreciable “safe” level essentially removing respiratory droplets as a carrier for virus such as COVID19. Once the door is closed, according to our CFD calculation, the SPC curves should relax with an exponential decay until the air quality is deemed “excellent”. Inventors have utilized the EPA's 50 (AQI) units is this determinative threshold. The exponential decay is supported by the transport diffusion theory of particles in a closed room (with the inflow/outflow boundary conditions provided from the HVAC system).²²

By fitting the exponential decay to the SPC curve in that time window, inventors eliminate the noise in the measurement, check that the door has not been open, and provide the air quality turnover time to reach the desired level of excellent air quality before the introduction of a subsequent patient for procedure or introduction of a cleaning crew. Once this SPC is leveled (under the 50 threshold), the current cyberinfrastructure can alert staff and/or management that the room should be ready for the next patient.

The present invention is designed to optimize an infrastructure usage, such as a procedural room in a hospital setting, that goes through the cycle: (1) room empty with door closed—(2) room open for preparation—(3) room in use for the procedure—(4) room cleaned for next procedure—(1) room empty with door closed—which is then repeated [Garbey2020a]²³. It is a stated goal of the present invention to provide a system that communicates, in real-time, air quality data to manage the risk of airborne disease due to aerosols wherein large quantities of small particle aerosols (less than 2.5 microns in diameter) are generated by surgical procedures exposing medical staff in that procedure room to the viral load of a patient and/or expose the patient from prior surgical suite patients [Garbey 2020b].²⁴

The present invention is directed toward improving the turnover process in a procedural room from both the perspective of safety and efficiency of surgical procedures constituting high aerosol generating procedures^{25,26}. Through data derived from a workflow application in a gastroenterology clinic treating a large number of representative patients with gastroenterology (GI) procedures such as colonoscopy, esophagogastroduodenoscopy (EGD), or Endoscopic Retrograde Cholangiopancreatography (ERCP), inventors were able to utilize data from those operations, each creating large quantities of small diameter particles in a surgical setting, in order to develop an air quality monitoring system, over and above theoretically supported HVAC (Heating, Ventilation and Air Conditioning) systems, for the mitigation of infective agent exposure during surgery.²⁷

In the case procedures are conducted on a positive, pre-symptomatic or asymptomatic COVID19 patient (or has received a false negative determination), contamination by airborne particles generated by the procedure itself becomes a serious concern for healthcare staff as well as other patients within a facility. Manifestly, the risk for contamination and infection resides with both the medical staff, present during and after the procedure, requiring those individuals to wear appropriate personal protective equipment (PPE), as well as patients, within a facility and later treated patients. Equally, all subsequent populations (medical staff, patients and custodial staff) run an appreciable risk of exposure if the air contamination has not been properly filtered and eliminated by the Heating, Ventilation and Air Conditioning (HVAC) system as measured by acceptable amounts of potentially infectious particularized matter. It is in the contemplation of inventors to utilize the present invention to monitor, test, calibrate, better utilize and support HVAC systems with a clearer understanding of air quality within a closed space.

The present invention provides for a system, device and method, for real-time evaluation of the length of time a room is required to be closed (sealed) after each procedure in order to receive an excellent air quality level (described below) to mitigate the risk of airborne contamination to subsequent patients undergoing surgical procedures as well as all healthcare staff. By continuously monitoring digital particle signals (providing procedural room air quality state), as well as flexible endoscope state, surgical implement state, door conformation, location of patient staff and equipment (through electronic field monitoring and tagging) and motion sensing, among other signals. It is further contemplated by inventors that the present system may be used in any closed space, of sufficiently small area and volume, where, so long as the space is amendable to closure (small enclosures, ATM vestibules, enclosed atriums, sufficiently small restaurants etc.), the present invention has applicability. Inventors also envision a determination of cumulative exposure to airborne particulates wherein an individual, given an identifiable length of exposure, may know the risk of ‘total’ pathogen exposure over time.

The system, apparatus and method of (1) reducing exposure to aerosolized particulate pathogens thereby (2) safely increasing surgical procedure throughput, (3) increasing surgical flow rates and (4) improving surgical suite turnover times (i.e., limiting non-productive facility space utilization) through air quality monitoring is accomplished through implementation of best practices and insuring air quality reaches an “excellent level” (50 or less described below) in order to provide an acceptable small particulate level for the elimination of potential virus carrying vehicles and mitigation of disease through removal of aerosolized droplet vectors.

For the purposes of the present invention an “excellent level” of air quality is expressed through an AIR Quality Index (AQI) defined wherein as (i.e., the size range encompassing the majority of ‘small particle’ focused biological materials), and the primary focus of the present invention, method and system of use, are expressed through a Small Particle Count (SPC) (as evidenced through a relative AQI) below 50.

### AQI: US

AQI or Air Quality Index varies by country but routinely measures specific pollutants (ground-level ozone, particulate matter in the 0.5/μg/m³ and 10 μg/m³ range, carbon monoxide, sulfur dioxide, and nitrogen dioxide) and resulting in categories roughly equated to a numerical value (0-500) wherein 0 is deemed “excellent” and ranges approximating 50 or less are considered “good”. The US has a total of 6 categories indicating increasing levels of threat or concern.

Aiding in the ease of reference and interpretation, inventors have maintained the color-coded designations of the US Environmental protection Agency (EPA) wherein green (0 to 50) is good (“excellent”), yellow (51 to 100) is moderate, orange (101 to 150) is unhealthy for sensitive groups, red (151 to 200) is unhealthy, purple (301 to 300) is very unhealthy, and maroon (301 to 500) is hazardous. It should be noted, however, that the actual levels employed by inventors is binomial wherein, in all practicality, the designations existing are (a) green (below 50) and (b) non-green (above 50), safe or unsafe. Inventors reserve the right to designate other levels, ranges and criteria where warranted and should the need arise.

Where small particle count is measured by converting the Dylos® laser particle counter (i.e., converting from the particle count concentration of 0.01 cubic feet into the mass concentration of micrograms per cubic meter or μg/m3) and then mass concentration into AQI requires the below formula:

I=((I_high−I_low)/(C_high−C_low))+(C−C_{low})+I_{low}
I=the (Air Quality) index,
C=the pollutant concentration,
C_{low}=the concentration breakpoint that is ≤C,
C_{high}=the concentration breakpoint that is ≥C,
I_{low}=the index breakpoint corresponding to C_{low},
I_{high}=the index breakpoint corresponding to C_{high}.
   This calculation may also be accomplished through one of multiple calculation websites.^{28,29}

Ultimately, though, while not directly measuring pathogenic small molecules (e.g., virus, mycobacterium and bacteria), the AQI acts as a shorthand approximation of small particle air contaminants wherein the AQI proxy designates those (small) particles of greatest concern. Principally, small particles (those less than 2.5 microns) are the particulate of most interest for their role in pathogenesis as enumerated above. This Air Quality Index is representationally depicted in the chart below:

Further, inventor's postulate a newly developed device having inventive and novel features (described in detail below) which serves to determine particulate count with several functions remaining unaddressed or underaddressed by Dylos®. And while inventor's device exhibits several features which improve on AQI monitoring, the determination of particle count nonetheless corresponds to the present determinations and monitoring.

This present invention, system and method of use provides a rational workflow solution to allow for the monitoring of the smallest micron particles (2.5 microns or less in diameter) capable of extended airborne suspension and the greatest penetration into the respiratory tract. The determination of the ambient air content of these smallest pathogen harboring droplets correspondingly allows for a reciprocal determination of the disease transmitting capability of said air. The knowledge of the ambient air content allows practitioners to determine the smallest interval between clinic procedures, improve the clinic's efficiency and output, all while maintaining the safety of staff and patients.

### DETAILED DESCRIPTION

GI procedures are relatively short procedures that take between 10 minutes for EGD to 20 minutes for colonoscopy, on average. The turnover time is the elapsed time needed between two procedures inclusive of a procedure's termination, cleaning the surgical area and bringing in a new patient for a subsequent procedure. Because these procedures are minimally invasive, an efficient clinical process theoretically should take no more than 5 to 10 minutes for routine turnover time. Yet, in the process of conducting GI procedures, there exists the potential generation of a significant number of aerosols, including those potentially containing viruses, bacteria and other pathogenic matter (e.g., fungal matter). Moreover testing for pathogens, virus tests in particular, have limited utility and reliability.^{30,31} Consequently, GI clinics often implement longer turnover times than is required to make certain that the ambient air has been completely recycled in the procedural room—choosing to err on the side of caution with regards to patient and staff health.³² But, in a multivariable environment, it is often complicated to determine the precise timing of determining ‘if’ and ‘when’ an acceptable threshold has been reached.

Currently, turnover times are based on the CDC's recommendation of estimating how long it takes for the HVAC system to renew 99% of the air in the room.^{33,34} Using the formula of reported by the CDC Guidelines for Environmental Infection Control in Health-Care Facilities (2003, updated 2019)³⁵ and measurement of inflow/outflow flux, one can obtain a theoretical prediction of the time it takes for the HVAC system to renew 99% of the air in a room or space assuming perfect mixing conditions. Yet, perfect mixing rarely occurs and removal times may be extended in rooms or areas with imperfect mixing, turbulent flow or air stagnation. To overcome these limitations, clinics may use their own individualized HVAC formulas with an extremely high-level elimination rate up to and including 99%.

However, the present results, based on small particle count (SPC) using data gathered from over 1500 GI cases in a single clinical suite to understand the correlation between air quality and standard procedure types as well as identify the risks involved with HVAC systems in a clinical suite environment, show (a) the air quality turnover varies widely from one procedure room to another or, in certain cases, in the same or identical rooms and (b) HVAC efficiency in procedural rooms may operate inexactly to specifications from the model formula (where this formula is an over simplification of fluid mechanics and geometry). This model formula can be influenced in one of a number of ways by (1) room geometry, (2) equipment and equipment orientation and arrangement, (3) flow creating impediments through energy emitting equipment, (4) redirection of air flow based on surgical staff and movements, (5) distribution and redistribution of airborne particles at the moment the door is opened or closed and (5) re-circulation zones and turbulent flows which completely change air direction—all causing HVAC renewal percentages and times to vary considerably. FIG. 15 exhibits this variability and related time estimates, represented in the tested clinic outpatient center.

Historically, a majority of clinics use a standard model formula based on (1) room size and (2) measured inflow/outflow of their HVAC system to estimate what would be a “reasonable” amount of time needed to eliminate airborne particles which is tested annually, or at best bi-annually. As described above, to determine the time period between turnover (i.e., how many minutes are required for each procedural room to refresh 99% of its air between procedures when the door are closed), perfect air mixing is supposed and is not designed to fit the complex dynamic of small airborne particle transport and deposition that can potentially carry the virus in clinical conditions.

This is vitally important wherein subsequent patients undergoing a GI procedure may be exposed to pathogens (e.g., bacterial infective agents, fungal agents or viral antigens up to and including COVID19) generated by any previous patient or transmitted via airborne vapor droplets. Manifestly, molecules of small particle sizes (2.5 microns or less in diameter) capable of carrying various antigens, of which COVID 19 is a member, can rely on many biological and physical factors which increase such particles virulence and communicability including: (a) ability to remain airborne, (b) aptitude to travel long distances, (c) ability to penetrate surgical masks and (d) proclivity to embed further down the respiratory tract—as opposed to larger particle sized (>5 μm). This ability to infect can be further augmented with distance to the emitting source whereby those closer to the source experience concentrated aerosols at a much higher rate than those in further proximity.⁸

Currently, there are no inventions incorporating an understanding and contemplation of the multitude of highly mutable parameters which even approximates the present invention specifically for risk mitigation and efficient room turnover. Further, the present invention has the capability to remediate the shortcoming of the current HVAC-enabled system without directly modifying the HVAC system. Additionally, the present invention has the ability to operate in numerous surgical spaces, regardless of configuration or procedure type, to effectuate a more efficient and timely air quality recovery system.

While strides have been made to overcome the inadequacies of maintaining appropriate air quality in a surgical area, it remains evident that considerable failings remain in the field. It is the goal of the present invention to remedy these shortcomings as to allow better monitoring and management of the air quality in “real-time” of surgical spaces and to potentiate a system of improved understanding of the physical and biological actions of small particles within a confined space and functions and operations which can accurately predict small molecule dynamics and potential antigenic virulence. It is a stated goal by inventors to mitigate relative pathogenicity within a space by confirming and assuring actual adequate AQI (≤50) thereby providing a quantitative measure of confirming an acceptable level of air particles and lessening the calculatable time between procedures accurately within said space. And it is manifest that acceptable levels of particulates in determining air quality, while varying significantly from one case to another, is still far better than the theoretical prediction based on the 99% elimination threshold given by HVAC system model formulas.

On average, inventors estimate that even reducing the turnover time from 30 minutes to 20 minutes based on air quality may have dramatic effects by increasing the number of procedures during a shift (7:00 am to 3:00 μm) from 6 to 8 procedures or 8 procedures to 10 procedures, representing a 25% improvement on procedure throughput—all with a high percentage of confidence on the accuracy of gathered data. Inventors have validated our invention to improve turnover in a GI clinic from a large data set having a relatively large volume of procedures per day upon which our clinically significant determinations are based.

Consequently, the application of this invention can be extended to any surgery center, surgical suite, operating room, post op area, examination room, waiting room or related medical facility space in which the space may be closed or “sealed”. What is more, the present invention has equal applicability in any confined or cordoned space whether it is an office space, workspace, factory, workshop or essentially any space having a defined enclosure and a door or multiple doors. Clearly, the same invention can be used for any industry that uses. Duly, individual closed-station units, where repetitive procedures generate aerosol, inventors envision preferred embodiments where a computer vision sensor is a camera or a plurality of cameras equipped with an AI software can recognize when the procedure starts and ends, as well as traffic of personnel. Such industry processes, for example, can be the turnover time between unloading/loading passenger for international flights, between shifts where employees are in close proximity or meat processing units where staff operate in closed stations and have rotations.

Manifest here, though, and in the context of a surgical space, instead of monitoring the flexible endoscopic tower activity that is central to the GI procedure, anesthesia machines may be used to detect when and how long the patient has been intubated, or alternatively the endoscope view for a minimally invasive surgery that counts for half of the surgeries practiced in western countries. Alternatively, both may be tracked and used.

It is the goal of inventors to provide for an efficient and safe means to ensure the air quality of a given space, especially in the case of a surgical space, wherein the health of the medical staff and patients depend on such determinations. Consequently, we conclude that our invention, method of use and system may provide a safer and more efficient mechanism of more accurately handling procedure turnover times in a clinic.

### BRIEF DESCRIPTION OF THE DRAWINGS

While the novel features and method of use of the application are set forth above, the application itself, as well as a preferred mode of use, and advantages thereof, will best be understood by referencing to the following detailed description when read in conjunction with the accompanying drawings in view of the appended claims, wherein:

FIG. 1 depicts the cyber infrastructure.

FIG. 2 shows various system components.

FIG. 3 is a simplified device mounted on wall adjacent to door.

FIG. 4 illustrates a top view of device in FIG. 3.

FIG. 5 shows a top view of FIG. 4 with inlet and outlet valves.

FIG. 6 is a modular version of FIGS. 3-5.

FIG. 7 correlates procedure initiation and particle count with time in 3 procedure day.

FIG. 8 correlates endoscopic state and particle count with clinic time in a 3 procedure day.

FIG. 9 correlates procedure state and particle count with time in 5 patient, 6 procedure day.

FIG. 10 correlates endoscopic state and particle count with clinic time in a 5 patient, 6 procedure day.

FIG. 11 depicts various steps within a typical GI procedure and logarithmic particle count.

FIG. 12 illustrates graphs particle count in the first 5 minutes of a colonoscopy versus the particle count in the 5 minutes prior to the procedure.

FIG. 13 shows particle count in the first 5 minutes of a (esophagogastroduodenoscopy) EGD versus the particle count in the 5 minutes prior to the procedure.

FIG. 14 depicts a distribution histogram indicator of aerosol generation (mean SPC) for a colonoscopy during the first 5 minutes of the procedure versus aerosol generation (mean SPC) during the first 5 minutes before the procedure.

FIG. 15 depicts a distribution histogram indicator of aerosol generation (mean SPC) for a EGD during the first 3 minutes of the procedure versus aerosol generation (mean SPC) during the first 3 minutes before the procedure.

FIG. 16 is a Distribution of Indicator of aerosol generation for a colonoscopy after the initial phase of the procedure.

FIG. 17 is a Distribution of Indicator of aerosol generation for a EGD after the initial phase of the procedure.

FIG. 18 illustrates the mean and standard variation of the lambda value obtained from each fitting in each procedural room.

FIG. 19 represents air quality turnover time measured under clinical conditions.

FIG. 20 shows turnover time computed from the HVAC formula provided by the CDC website.

FIG. 22 represents a graphic user interface (GUI) providing room state and air quality turnover information in a GI center with 7 procedure rooms.

FIG. 23 provides a legend of GUI determinates.

FIG. 24 is a multiview, multiscreen dashboard GUI showing several views.

And while the invention itself and method of use are amendable to various modifications and alternative configurations, specific embodiments thereof have been shown by way of example in the drawings and are herein described in adequate detail to teach those having skill in the art how to make and practice the same. It should, however, be understood that the above description and preferred embodiments disclosed, are not intended to limit the invention to the particular embodiment disclosed, but on the contrary, the invention disclosure is intended to cover all modifications, alternatives and equivalents falling within the spirit and scope of the invention as defined within the claim's broadest reasonable interpretation consistent with the specification.

### Experimental Evidence and Data

### Aerosol Generation³⁶

Inventors collected time series data over a period of approximately 50 consecutive clinical days in seven procedural rooms simultaneously. Overall, inventors collected a dataset with 1213 colonoscopies and 594 EGD.

Initially, to begin, inventors analyzed the aerosol generated by the procedures with our SPC measurements (described below). For those 301 patients who had a hybrid procedure (i.e., an EGD immediately followed by a colonoscopy) in our dataset, the interval of time between the EGD and colonoscopy that follows is so short that the SPC during the colonoscopy might be impacted by the EGD. Thus, we removed the colonoscopies from these hybrid procedures in our study. For the 912 colonoscopies left and 594 EGD, we compute the average number of particles at different steps:
Step 1: The 5 min window prior to the procedure, when the medical team is already in the room and the door closed, with the air quality average during the whole procedure, denoted Dₚᵣₑ
Step 2: The first 5 min of the colonoscopy, and respectively the first 3 min of the EGD procedure, denoted Dₛₜₐᵣₜ
Step 3. The rest of the procedure, denoted D_{proc}

For those procedures generating aerosols, inventors expect that the average during Step 2 will be larger than during Step 1. Using inventors' previous observation, inventors' hypothesis is that in the first few minutes of the procedure (Step 2) the patient reacts to a foreign body inserted into patient's gastric path with more vigor than during the rest of the procedure. To verify the hypothesis, inventors also computed the average SPC during the procedure after the initial phase mentioned above (Step 3).

Next, inventors analyzed the air quality of the procedural room during the turnover time between two patients. To avoid any interference and minimize the noise in the dataset, inventors imposed a number of criteria to restrict our analysis to a subset of “procedure turnover” SPC signals. Inventors assume:
1. The procedural room goes through cleaning during a small interval of a few minutes right after the procedure is done.
2. The door is closed and staff do not open the door of the procedural room until the air quality is excellent, i.e., SPC has been below 50 u_{d} for 5 min.

Inventors use signal analysis on the SPC curves to keep only the signal from the Dylos® sensor that would correspond to this hypothesis. Firstly, inventors keep only the data set with a sharp peak of particle counts occurring soon after the procedure is complete. Secondly, as soon as the door of the procedural room is closed, inventors observed that the particle count decreases in the first approximation exponentially. Inventors expect this exponential decay from the standard diffusion convection process in a closed space, with inlet/outlet flow corresponding to the HVAC system, that should be a dominant factor. Thirdly, inventors restricted themselves to an interval of time that leads to a decay of particle count below 50 u_{d}. Using all three criteria, inventors found a dataset of 199 turnover air quality signals that inventors found capable of accurate analysis. The benefit of this approach is that inventors were able to properly compute the exponential decay of particle counts at turnover to check inventors' hypotheses without external interference, for example, inventors were able to show that large particle count decays more quickly than small particle count, as expected. Inventors were also able to compare how fast the HVAC system cleans out the air for each procedural room and demonstrate that the formula presented by the CDC in has strong limitations.³⁷

Inventors showed the ratio of the mean SPC during the first 5 min of a colonoscopy divided by the mean SPC during the 5 min prior to the colonoscopy procedure

$${R}_{col} = \frac{\begin{matrix}D col \\ start\end{matrix}}{\begin{matrix}D col \\ pre\end{matrix}} .$$

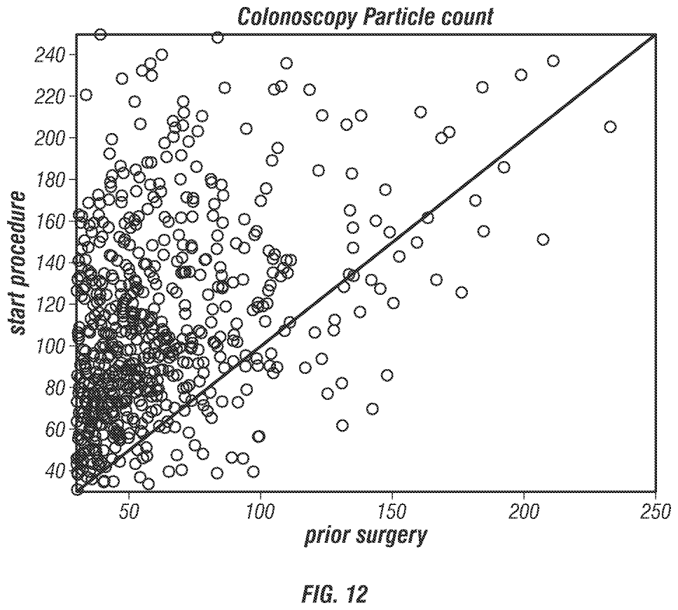

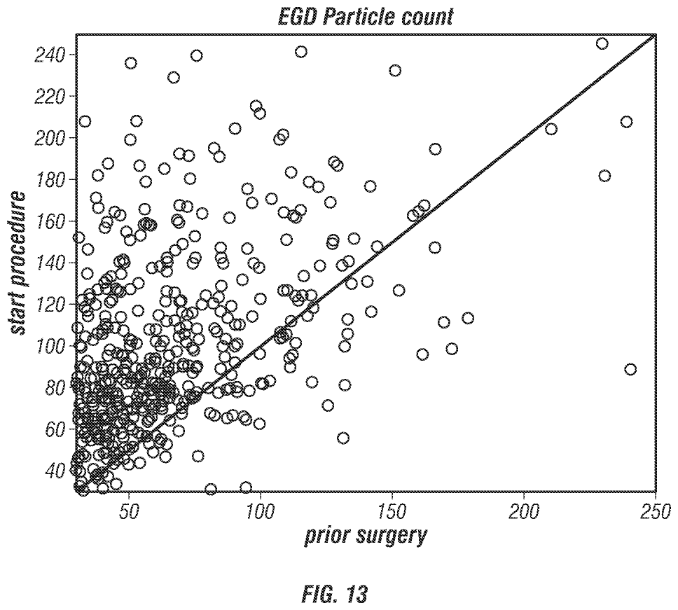

Usually during this time window (Step 1), the patient is already inside the procedural room along with the medical team, composed of the gastroenterologist, a registered nurse, anesthesiologist, and a technician who helps with the endoscope. Inventors use this time window as a baseline to rate the particle count generated from the procedure itself, since the door is closed and the number of people who are in the procedural room typically stays the same. Inventors observed that, in the vast majority of the cases, this ratio is above the y=x line. FIG. 12 gives the histogram of the ratio R_{col} and shows a biased distribution with 2 as its maximum. According to inventors' interpretation, these figures show that colonoscopy procedures generate a significant level of aerosol post procedure start.

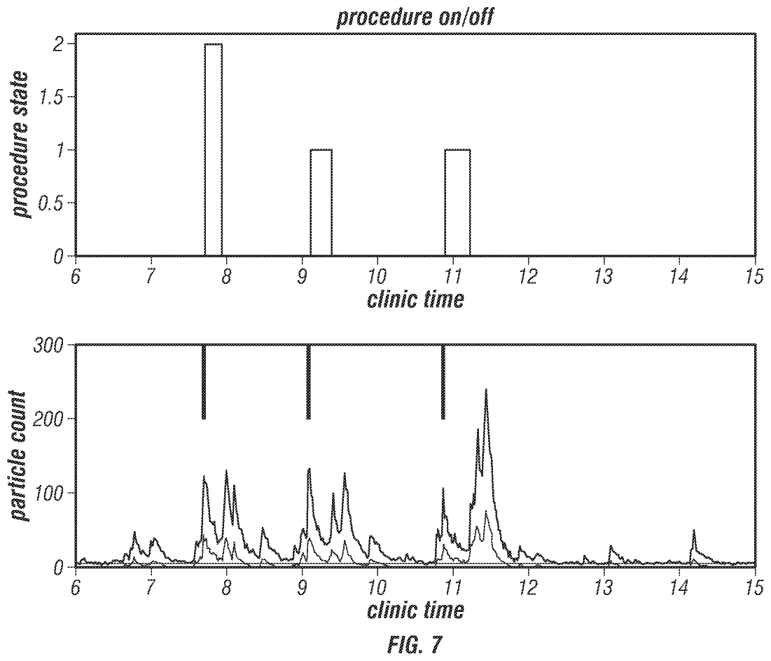

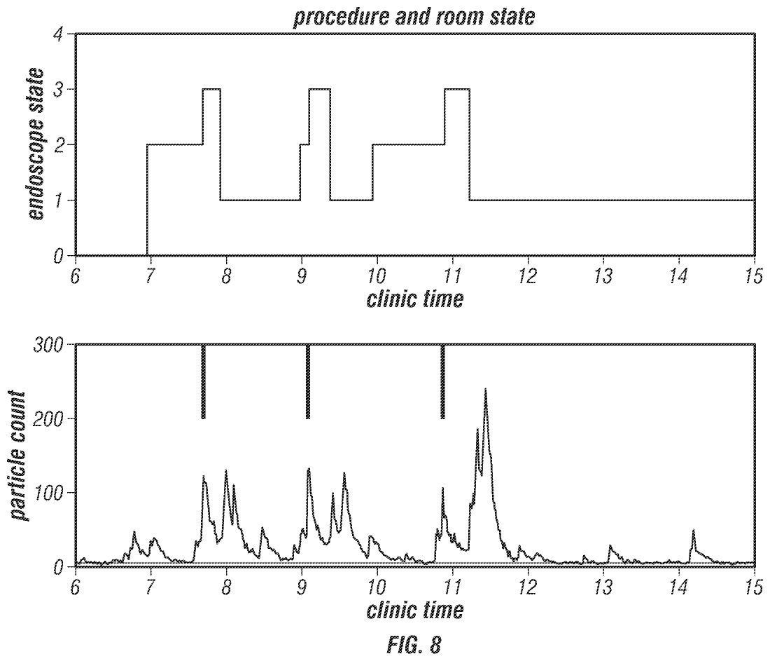

FIGS. 7 and 8 report on similar results with EGD procedures with

$$Rcol = \frac{\begin{matrix}D egd \\ start\end{matrix}}{\begin{matrix}D egd \\ pre\end{matrix}} .$$

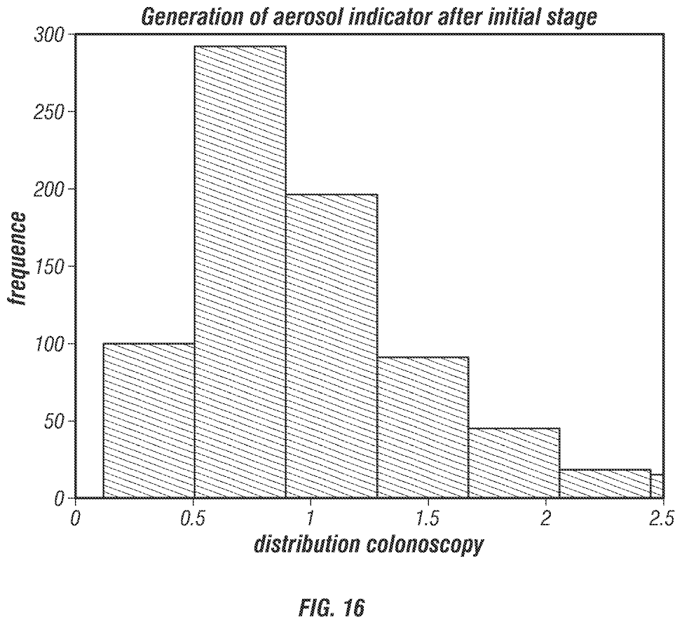

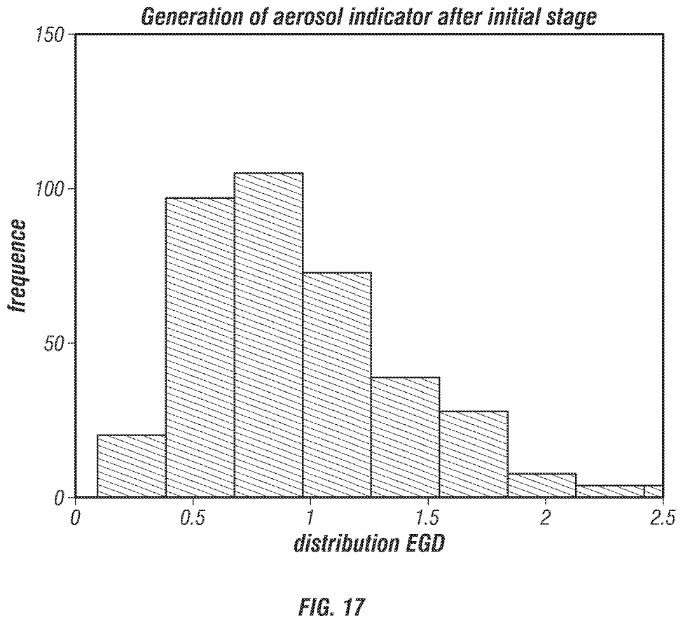

Because an EGD takes about half the time of a colonoscopy to complete, inventors look at the ratio of the mean SPC during the first 3 min only, Dₛₜₐᵣₜ^{egd}, versus the mean SPC during the 5 minutes prior to EGD procedure Dₚᵣₑ^{egd}. As shown, an EGD generates fewer particles in proportion to a colonoscopy during the initial phase of the procedure when accessing the anatomic region of interest with the endoscope. FIG. 16 gives the distribution of the ratio P_{col} of the mean SPC during the rest of the colonoscopy procedure (Step 3), and FIG. 17 or the EGD procedure P_{egd}, versus the mean SPC prior to the procedure. The mean of that ratio over all colonoscopy procedures is 1.08, and 0.98 for EGD procedures. Of these procedures, 40% of them generate small particles during the procedure past the initial phase.

The risk of virus carried by small particles coming from the patient is therefore higher during the first few minutes of a colonoscopy and an EGD. However, this risk remains during the entire procedure for almost half of the procedures observed. This measurement supports the fact that PPE is essential for the staff.

Once the procedure is done and the surfaces are cleaned by the janitorial teams, one needs to make sure that most airborne particles generated by the patient procedures are evacuated by the HVAC system. Inventors discuss next the result of their clinical study.

Inventors systematically fitted the following exponential decay function to the SPC curves in the time window of the turnover time between procedures, which is bounded by the highest peak value of the SPC (corresponding to the quick cleaning period of the room) and the lowest value that is below 50 u_{d}, marking the excellent air quality level.

A·exp(−λ(t−t₀))

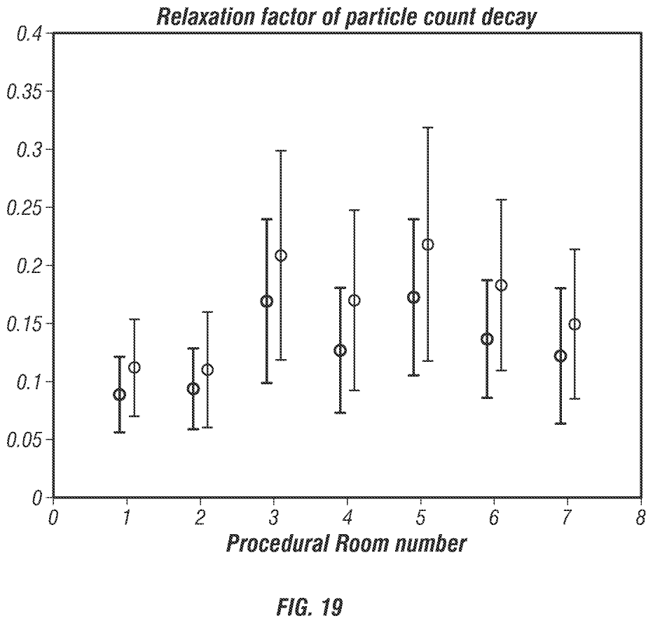

A and λ obtained with a least squares method. In principle, the procedural room door is closed during that time because the SPC in the hallway is much higher in our observation. FIG. 19 shows the mean and standard variation of λ value obtained from each fitting in each procedural room. Inventors present the same calculation but for the large particle count using the same time interval. The λ value for small particle is smaller than the λ value for large particles which means that the relaxation time for small particles is longer than the relaxation time for large particle, as expected, wherein large particles are eliminated more quickly than the small particles, which confirms the relative higher risk for SPC than Large Particle Count (LPC) and the importance of monitoring its value.

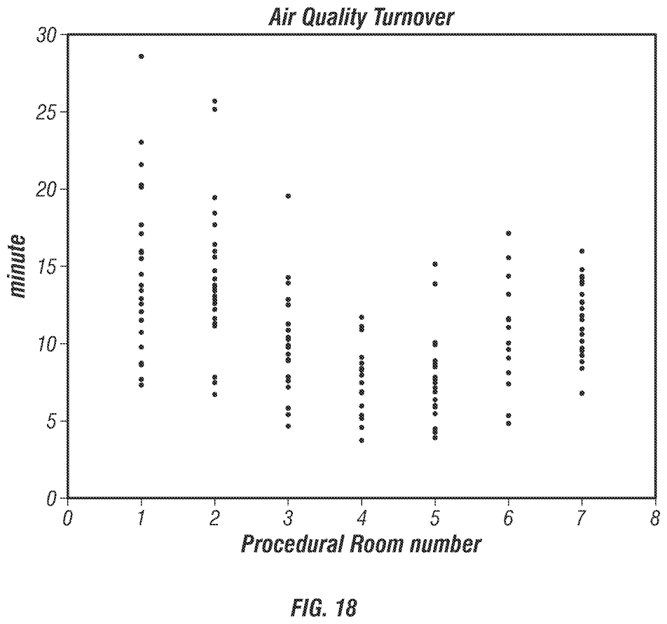

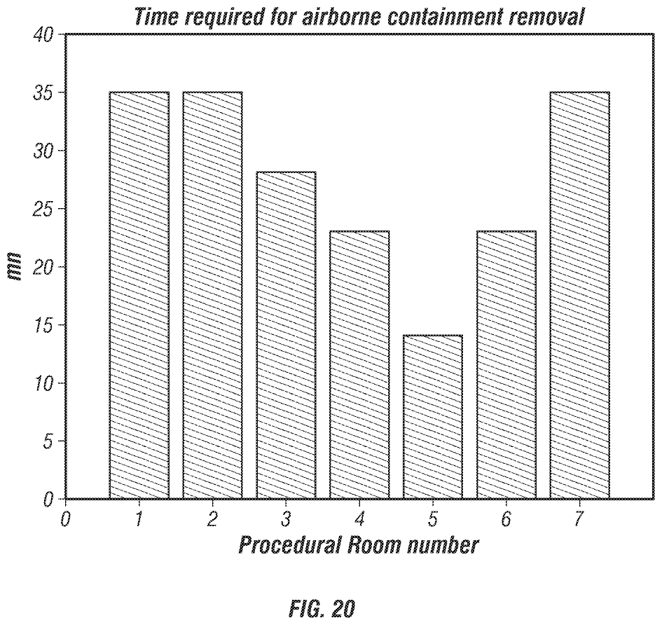

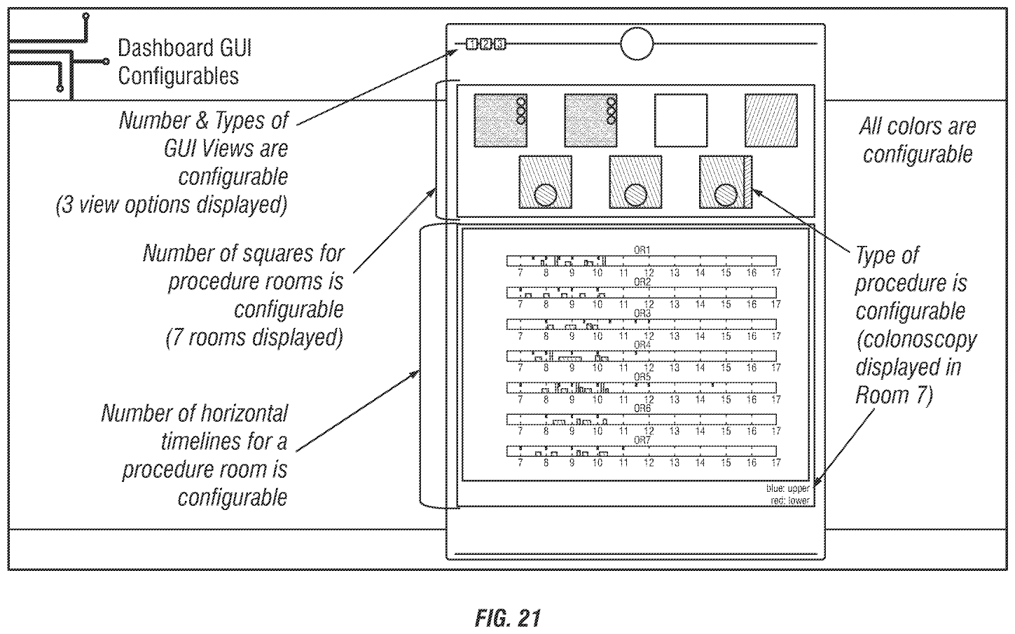

Inventors also note that the decay factor depends on the procedural room itself. As a matter of fact, each procedural room may have a different structural size and inflow/outflow boundary conditions from the HVAC system. Using the above HVAC formula, reported by the CDC guidelines for environmental infection control in healthcare facilities (2013) and measurement of inflow/outflow flux, one can obtain a theoretical prediction of the time it takes to the HVAC system to renew 99% of the air in the room. FIG. 20 reports on this time estimate. Inventors then made a qualitative comparison between this theoretical estimate and the theoretical estimate based on a gross approximation of mass transfer of the air. Inventors focus first on the relative comparison between each room air quality turnover to see if results in FIGS. 18 and 20 (which are correlated). The relaxation factor inventors found for Rooms 1 and 2 is about the same order and the smallest of all procedural rooms observed. Accordingly, Rooms 1 and 2 in FIG. 20 have the largest turnover time to renew the air. However, according to our measurements, Room 7 renews air more quickly than the time given by the model's formula. Rooms 4 and 5 seem to have a similar air quality turnover, while, in the theoretical estimate of FIG. 20. Room 5 has the best performances of all. Overall, inventors observed a weak correlation between the theoretical prediction³⁷ in and our measurement of air quality turnover. Inventors note that the computed λ value varies significantly: according to computational fluid mechanics, the air quality renewal is dependent of the initial condition of air quality distributed in the three-dimensional volume of the procedural room.³⁸ This initial condition may depend on a number of factors related to the dynamic of people leaving the procedural room, door motions, and the amount of aerosol that has been generated.

From this practical perspective, the most important factor is knowing how long it takes for the SPC to go back to an excellent air quality level that approximately corresponds to the baseline prior to any procedure. FIG. 18 shows that, on average, after the procedural room is closed, it takes about 10-15 min to regain excellent air quality levels. While this is a much lower turnover time than the model's prediction, the air quality turnover time associated with the measurement of SPC varies based on the room conditions, when the procedure is finished, and if the cleaning has been done. Therefore, the HVAC computation provided by The American Conference of Governmental Industrial Hygienists, Inc. (ACGIH) can be approximated to be very close to the worst-case air-quality turnover observed by inventors.

Therefore, on average, inventors estimated that reducing the turnover time from 30 to 20 min based on air quality may increase the number of procedures during a shift from 7:00 a.m. to 03:00 p.m. from 8 procedures to 10 procedures, which represents a 25% improvement on procedure throughput.

The benefit of inventors' cyber-physical infrastructure is that it continuously gives the SPC in every procedural room, giving clear information to healthcare staff helping them minimize risk exposure. By introducing a concept of air quality turnover that follows each cleaning step of the room, inventors' system helps to ensure that the door of the procedural room is properly closed as soon as possible, after patient admission and patient egress, in order to let the HVAC system renew the air efficiently. As noted earlier, any door opening or lack of sanitizing the room in time is easily detected by our system. Inventors developed in the present system a phone application to quickly and easily support the staff in their daily work. Over time and usage, the system has become an autonomous, best-practice process that supports healthcare teamwork

Today, most clinics use a standard model formula based on room size and measured inflow/outflow HVAC to estimate what would be a reasonable amount of time needed to eliminate airborne particles. This is important because any healthcare professional should always ensure that the next patient going into a GI procedure will not be exposed to COVID-19, or any other pathogens, generated by any previous patients. As is known, asymptomatic patients might not be detected adequately by today's testing.

Too, perfect mixing usually does not occur. Removal times will be longer in rooms or areas with imperfect mixing or air stagnation. To overcome these limitations, clinics use their own HVAC formulas with a very high level of elimination rate such as 99%. However, inventors' results, based on SPC, shows that the air quality turnover varies widely from one procedure to another in the same room, and that HVAC efficiency in procedural rooms may not perform exactly as expected from the model formula, which is an oversimplification of fluid mechanics and geometry. Their model formula can be miscalculated by: (1) filling a room with numerous pieces of equipment that generate energy; (2) the distribution of airborne particles at the moment the door is closed to let the HVAC renewal work may vary considerably; and (3) re-circulation zones and turbulent flows may completely change the results.

### Transport and Diffusion of Hazardous Airborne Particles²³

Surgical smoke can act as a carrier for tissue particles, viruses, and bacteria. Likewise, those same particles can be on the same order of small particles (2.5 microns and under) which have been discussed as having a high propensity to convey viral pathogens. To quantify airborne transmission from a physical point of view, inventors consider surgical smoke produced by thermal destruction of tissue during the use of electrosurgical instruments as a marker of airborne particle diffusion-transportation. Surgical smoke plumes are also known to be dangerous for human health, especially to surgical staff who receive long-term exposure over the years. There are limited quantified metrics reported on long-term effects of surgical smoke on staff's health. The purpose of the below is to provide a mathematical framework and experimental protocol to assess the transport and diffusion of hazardous airborne particles in every large operating room suite. Measurements from a network of air quality sensors gathered during a clinical study provide validation for the main part of the model. Overall, the model estimates staff exposure to airborne contamination from surgical smoke and biological material. To address the clinical implication over a long period of time, the systems approach is built upon previous work on multi-scale modeling of surgical flow in a large operating room suite and takes into account human behavior factors.

To quantify airborne transmission from a physical-spatial perspective, inventors consider surgical smoke as a marker of airborne particle diffusion-transportation emitted from the surgical table area. Surgical smoke is 95% water or steam and 5% particle material and therefore surgical smoke can act as a carrier for tissue particles, viruses, and bacteria.³⁹ surgical smoke carries Ultra Fine Particles (UFP) as small as 0.01 microns, which are able to bypass pulmonary filtration, and small particles up to several microns.⁴⁰

Inventors advance a rigorous multi-scale computational framework to address these questions and use measurements of diffusion-transportation of surgical smoke particles with off-the-shelf portable sensors to calibrate the model. Further inventors seek to quantify the level of exposure in order to estimate the corresponding viral load in part. Transport and diffusion mechanisms are very effective for UFP to travel a long distance from the source in a short period of time. Clinical environments are too complex to model with the traditional modeling method of airflow and particle transportation because both the source intensity of surgical smoke⁴¹ as well as the mechanism of propagation via door openings⁴² are largely dominated by human factors.

The geometric complexity of the infrastructure and of the heating, ventilation, and air conditioning (HVAC) system limit the capability of Computational Fluid Dynamics (CFD) to predict indoor air quality and health and droplet behavior depends not only on their size, but also on the degree of turbulence and speed of the gas cloud, coupled with the properties of the ambient environment (temperature, humidity, and airflow).

It is the above challenges inventors address through a mathematical framework and experimental protocol to assess the transport and diffusion of hazardous airborne particles in any large OR suite wherein human behavior factors are taken into account by using a systems and cyber-infrastructure approach coupled with a coupled to a multi-scale modeling of surgical flow in a large OR suite. Overall, the model estimates staff's exposure to airborne contamination, such as surgical smoke or biological hazard. Validation is provided by a network of wireless air quality sensors placed at critical locations in an OR suite during the initial phase of the surgical-suite-specific study.

A step-by-step construction of the model scaling up from the OR scale to the surgical suite scale will be presented wherein the present model integrates the transport mechanism occurring at the minute scale with the surgical workflow efficiency simulation over a one-year period. To assess potential contamination from one OR to another, the extent of the propagation of surgical smoke in the area adjacent to the OR were checked.

To simulate the airflow and dispersion of surgical smoke, an OR that is representative of a surgical suite was utilized and measurements for calibrations and verifications were conducted in an unoccupied OR. Airflow was assumed to be turbulent and was modeled using the k-e turbulence model, taking into account gravity to introduce the Boussinesq approximation in the Navier-Stokes equation. It is the most common model for indoor airflow simulation in which the turbulent kinetic energy k and turbulent dissipation rate e are modeled. Too temperature and pressure were measured and placements of surgical were noted as obstructions. Pressurization is a key factor in controlling room airflow patterns in a healthcare facility where positive pressurization is used to maintain airflow from clean to less-clean spaces (positive pressure of about 8 Pa).

For the hallway, a uniform inflow boundary condition of 0.1 m/s was imposed in order to take into account the anemometry measurement mentioned above. This upstream boundary condition is completed by a free outlet boundary condition at the other end of the hallway. To be as realistic as possible, the two existing inlet vents' boundary conditions were respectively added for the inlet vent located on the ceiling and the other inlet located close to the entry door of the next OR, both with a velocity of 1.2 m/s. The goal of the CFD simulation is to be able to assess the rate of pollutant leaving or entering the OR depending on if the door is closed (0°) or completely open (90°). During the observation of the clinic work, OR doors would sometimes stay open for several minutes and for different reasons. The doors of the ORs downstream were left open in order to check the potential inflow of pollutants from the other ORs.

To validate the model, measurements were done of velocity flow and of the concentration of particles at various locations that were close to specific regions of interest—the door-frame location in particular, measurements are reported in the Result section. Next, an upscale model was presented that will use the present CFD simulation to verify the key parameter values, especially relating to transmission parameters between ORs and the hallway.

To represent tissue, electrosurgical energy was delivered on the surface of two pieces of pork meat, each 2 cm thick, placed on an OR table and three types of energy delivery systems were compared: electrosurgery (conduction) via the Covidien ForceTriad monopolar device (Medtronic, Minneapolis, MN, USA), ultrasonic (mechanic) with the Ethicon Harmonic Scalpel P06674 device (Ethicon Inc., Somerville, NJ, USA), and laser tissue ablation with Erbe APC (Argon Plasma Coagulation) 2 device (Erbe Elektromedizin GmbH, Tübingen, Germany). The measurement was conducted by several laser particle counters from Dylos® Corp (Riverside, CA, USA) placed at various distances from the source. Said sensors gave an average particle count every minute in a unit system with units u_{d} that correspond to 0.01 particles per cubic foot or 0.00028 particles per cubic meter (1 cubic foot=0.0283168 cubic meter) wherein the Dylos® sensor whose output is highly correlated (r=0.8) to a “ground true” measurement provided by a high-end system such as the Sibata LD 6S (Sibata Scientific Technology Ltd., Tokyo, Japan) that is claimed to be accurate within 10% in controlled laboratory conditions—ideal for accurate measurements at lower concentrations. Said sensors were set up to track particles of small size in the range from 0.5 to 2.5 microns, which are the sizes of biological material. The results were checked systematically by comparing the measures of several sensors at the same location to show consistency, as well as checked that the particle count lowers back down to nearly zero in a clean-air room with AC equipped with High Efficiency Particulate Air (HEPA) filters. Each experiment was started from an initial clean-air condition of a small particle count, fewer than 50 units, which is much less than the number of particles counted during energy delivery. It took about 6 min to reach the initial clear-air count after each experiment.

For each experiment, the concentration increased to a maximum after a short time delay s from the time the energy was delivered; this delay depends on the distance to the source. The concentration then exponentially relaxes to zero in time. Consequently, the model of source dispersion is an exponential function as follows:

ƒ(t)=Ae^{-ρ(t-s)}

The least squares fitting technique was used to interpolate the data with this function. The amplitude of the source A, the delay s on particle diffusion and transport to reach the sensor and the rate of “diffusion decay” ρ>0 were identified. The accuracy on s, which measures the time interval between the source production and the peak of the signal, cannot be faster than one minute since the sensor only works at a one-minute timescale. A delay of s≤1 was found to be a good approximation for all three energy devices. Each experiment was done 4 to 5 times depending on the variability of the results. Therefore, about 24 to 30 data points were available to identify the parameters A, s, and ρ for each energy device that was tested.

This set of experiments, as opposed to the previous one, were carried out in a large OR suite late at night and on weekends when the ORs were empty and had clean air with high-efficiency HVAC. A hairspray product (Lamaur Vitae, unscented) was used as the marker and sprayed for a duration of 1 to 2 s to track its small particles while keeping the same positions of the Dylos® systems. The experiment first tested the propagation in a closed-door OR with the source above the OR surgical table. The spray nozzle was held facing the near-vertical direction, pointing to the ceiling. A distribution of sensors as was used.

The initial observation was that all four sensors distributed along the central line of the whole OR space were getting a particle count of the same order of magnitude after an average of 15 s. The mixing of particles was quite extensive within a minute by reason of the HVAC input/output design in the OR, and that the concentrations on each sensor quickly relaxed to zero. This observation is also coherent with the results of the CFD model of the flow circulation described above.

A method identical to the previous one was used to identify the key parameters A, s, and ρ characteristic of the dispersion of hairspray in the OR.

The model for OR diffusion of particles is then:

$$\frac{d}{dt} Q ( t ) = - {ρ}_{OR} Q ( t ) + S ( t = 0 ) , t >= 0$$

where Q denotes the global concentration of particles in each OR, S(t=0) denotes the source production that is non zero at time zero and ρ_{OR} denotes the diffusion decay inside the OR. This simple Ordinary Differential Equation (ODE) model provides an average of particle concentration in the OR at the minute timescale.

A first-order implicit Euler scheme with a time step dr of one minute was used as follows:

(d/dt)Q(tₙ₊₁)≈Q(tₙ₋₁−Q(tₙ)/dt,Q(t₀)=S(t=0)

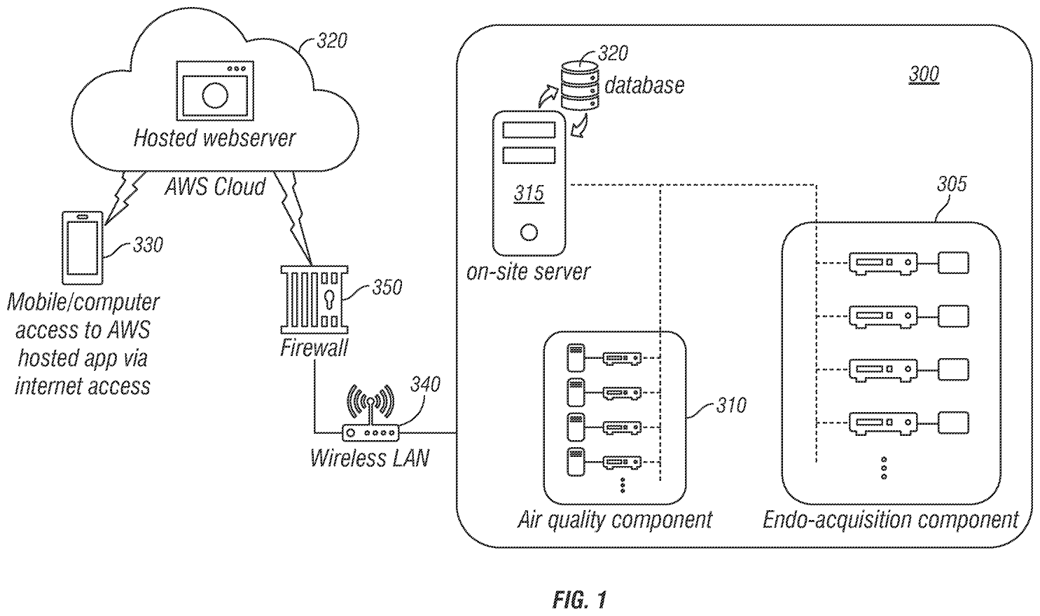

An entirely similar technique is used to describe the dynamic of particle diffusion and transport in the hallway, except that the hallway is discretized as a one-dimensional structure of consecutive hall blocks located at the same level as the OR block. In this part of the experiment, the source is set in the hallway. As noticed earlier, there is a slow but significant air flow speed v₀ in the hall, pointing in the direction of the main entrance of the surgical suite. Naturally, the high pressure of the OR is designed to drive the airflow out and the front corridor seems to be a significant outlet. On the opposite end of the hall, situated on the left of the map, FIG. 1, this velocity is close to zero. It is assumed that v₀(x) is an affine function, with a linear growth from 0 to 0.1 m/s at mid-hall, and a constant value beyond. The model of hallway diffusion of particles is then:

$$\frac{D}{Dt} P ( t ) = - {ρ}_{OR} Q ( t ) , t >= 0 ,$$

Where D/Dt denotes the total derivative

$$\frac{\partial }{\partial t} - {v}_{0} \frac{\partial }{\partial x}$$

using the x coordinate system in the one space dimension hall model.

To assess the transmission of particles from an OR to the adjacent hallway with closed OR doors, the same experiments were run with some of the sensors placed in the hallway either facing the closed door or sitting at a location in the hallway. As a matter of fact, the door of the OR is not perfectly sealed due to the difference between the pressure inside the OR and the lower pressure in the hallway, a significant airflow with velocity around the order of 1 m/s exists at the gap located between the door's edge and the door frame. A similar technique is used to represent the diffusion coefficient as well as the delay s that is now interpreted as the time it takes for the particles to flow from the OR to the hallway right outside the door.

Finally, an entirely similar approach is used to get the transmission in the compartment model when the door of a specific OR is wide open. In such cases, the gradient of pressure between the OR and the hallway nearly vanishes. At the doorstep, inventors observed buoyancy-driven effects due to the difference in temperature between the OR (cold air) and the hall (warm air). There is a convective flow exchange with cold air at the bottom going out of the OR and hot air at the top going into the OR. During our experiment with particle sensors, inventors were able to validate the propagation of aerosol traveling into the OR from outside when the door is left open. With the CFD model and taking into account the gravity, inventors simulated the contamination by adding a source of CO₂ from the inlet at the beginning of the hallway and keeping the door open. It took 18 s for the gas to reach the door and start contaminating the OR. This proved the importance of keeping the door closed to maintain the positive pressure in order to control the contamination rate and nosocomial propagation in the OR suite.

Now, the simple compartment-like model to monitor, in time and in space, the diffusion and transport of particles with intermittent source production in each OR was be assembled. Such a source of pollutants corresponds to either the use of some chemicals or the use of electrosurgical instruments during surgery. The goal is to get the average rate at which the staff working in the OR suite is getting exposed to particle concentration emanating from surgical smoke throughout the day. Potential propagation of particles that may carry biological material from one OR to another is also of interest.

As discussed earlier, the concentration was tracked in time and in space with a coarse time step of one minute. This time step scale is coherent with the measurement system used for particle counting. One minute is also roughly the time that the particles emitted from a point source next to the OR table need to transport and diffuse throughout the OR block once released. The compartment model computes the global concentration of the particles in each OR as well as in each section of the hall adjacent to the OR. These concentrations are denoted respectively Qⱼ(t) for OR number j at time t and Pⱼ(t) for the corresponding section of the hall.

The source of particles is denoted as Sj(t). In principle, Sj(t) should be non-zero for a limited period of time and follow a statistical model based on the different phases of the surgery and the knowledge of electrosurgical instrument used during a surgical procedure. The coefficients of decay are defined inside different parts of the model (ρ_{OR} and ρ_{Hall}) as well as the coefficients of transmission between these spaces (α_{OR} from the OR to the hall and γ_{Hall} for the opposite). β_{OR} represents the flow from the OR to the hall when the door is open. The frequency of door openings is following a statistical model based on where the surgery is at δⱼ^{door} is a function of time and is 1 if the door is open, 0 otherwise. The simulation of the surgery schedule uses data from the SmartOR project, which will be detailed later on. Only the door openings of the order of a minute will be counted and γ_{Hall}=β_{OR} will be assumed because the gradient of pressure between the OR and hallway vanishes.

The system model of marker transport-diffusion in the OR suite is as provided in reference 23 (Garbey 2020a), equations 4, 5 and 6.

Using these equations, the number of particles going from one OR to another can be separately counted. This number is expected to be very low—see “Results” section.

The model (4) and (5) is not a standard box model. First, the source term has a delay built-in to simulate the transmission conditions observed. Second, Equation (5) is a PDE, more precisely a linear transport equation. Third, most of the coefficients are stochastic, especially those related to door openings and sources that are linked to human behavior. Because the system of equations is linear, the superposition principle has been implicitly used to retrieve each unknown coefficient from the experimental protocol.

To describe the surgical flow model more precisely in order to provide an accurate description on how inventors managed to compute the source term Sⱼ(t).

or each of the standard OR stages of the surgery, an attributed State value is given as follows:
Phase 1: anesthesia preparation label as State=1.
Phase 2: surgical preparation to access as State=2.
Phase 3: surgery procedure as State=3.
Phase 4: surgery closing as State=2.
Phase 5: ending anesthesia as State=1.
Phase 6: room in the process of cleaning or free as State=0

The type of airborne marker expected to release depends on those State values. For example: In State 0, cleaning crew uses a lot of chemical products that quickly evaporate in the OR. Similarly, a different type of sterilization product is used to prep the patient in State 1. In State 2, cauterization is often used for a short period of time. In State 3, various phases of the surgery will require energy delivery instruments to cut tissue and access specific anatomy or tumors.

A stochastic model of energy delivery is used that consists of delivering short time fractions of energy in several consecutive minutes. The parameters of that model are: the frequency of energy delivery denoted ƒ, the duration of the impulse denoted ξ, and the number of repetition r. A uniform probabilistic distribution of events is used within these intervals of variation for each parameter.

Both the detection of door openings and a patient bed coming in and out were provided by the sensors of the cyber-physical infrastructure. A stochastic model of door openings was used based on a uniform frequency of door opening during surgery, even though this distribution is non-uniform in practice and tends to concentrate at the beginning and the end of a case.

The model of air pollution in the surgical suite will first be tested with a simplified model of surgical flow as follows: to provide the timeline of events, the model assumes there are three surgical procedures in each OR. The timeline of each surgery will be such that: Phase 1 and Phase 5 last 12.5 min±5 min, Phase 2 and Phase 4 last 15 min±5 min, Phase 3 is the surgery itself that lasts 65 min±25 min. Phase 6 corresponds to a turnover time between surgeries that lasts 30 min±10 min. This simplified model of surgery scheduling has the correct order of time-length for each phase. Its simplicity allows a sensitivity analysis to run with respect to the key parameters of the indoor air quality model that can be easily interpreted.

The mathematical model of surgical flow is built upon observations and robust clinical data covering 1000 procedures with a noninvasive array of sensors that automatically monitor the surgical flow. To this end, several ORs were equipped with sensors that capture timestamps corresponding to the different states described in the previous section.

Overall, the model can simulate the OR status of a large surgical suite during any clinical day and can be run over a long period of time. The model is able to reproduce the statistic distribution pattern over a year of performance indicators: turnover time, induction of anesthesia time, the time between extubation, and patient exit. The model classifies the human factors impact and limitation of shared resources on flow efficiency. In the end, communication delays and sub-optimal OR awareness in large surgical suites have significant impacts on performance and should be addressed. This paper concentrates on the duration of surgery State 3 that corresponds to how long surgical smoke is generated, and how behavior inducing OR door openings are responsible in part for the spread of surgical smoke and other agents. The output of the ABM model of surgical flow coupled to the air quality model is the number of hours per year that staff gets exposed to surgical smoke in the OR and hallway. Various scenarios have been run related to the rate of adoption of vacuum systems for surgical smoke and OR door openings to discuss the influence of human behavior on those results.

The measured rate of particles generated by various energy sources, such as monopolar cautery, argon plasma coagulation (APC), and harmonic sources, are found by testing them in an OR space allocated to training, i.e. without patients. The unit used for the source of the emission is 0.01 particles per cubic foot; it gives the measurements an order of magnitude from ten to thousands by which they can be compared. Small particles are found in the range of 0.5 to 2.5 microns, which are the sizes of biological-material particles like viruses.

It was found that the mixing of contaminants from a burst source to the rest of the OR is reached within a minute; applying a simplified compartment model to describe the OR's contribution to pollutants using a time step of one minute became apparent. This upper-scale model described in the methodology section will be calibrated next.

The identification of the model parameters from the experimental data-set corresponding to the setup is explained below. The experiment was designed first, to assess the delay of pollutant transmission between the OR and the hallway depending on if the door was opened or closed. Second, to compute the rate at which pollutant concentration decreases an exponential decay was observed in the OR, which is consistent with the fact that diffusion is the main mechanism due to the small velocities present inside the model. However, the hallway behaves more like a duct with a combination of convection and diffusion running down the hallway. These measurements are consistent with the CFD simulation results shown previously. Fitting the simplified model to the controlled experiment with a single source of smoke, the coefficients of diffusion in the OR and in the hallway can be retrieved, as well as the convection velocity in the hallway.

The diffusion coefficient in the OR and the hallway are dependent on the HVAC system that is, by design, more effective in the OR than in the hallway. Therefore, the rate of decay in the OR is twice as large as the rate of the decay in the hallway. As reported before, the diffusion coefficient for the particle tracking setting is about the same for the spray source as it is for the monopolar or APC sources. The transmission condition with a closed OR door is not negligible: it is about 4 times less than with an open door.

The most important result is surgical smoke emitted in a single OR can rapidly reach the hallway within a minute due to the OR door opening and is diluted by a factor of roughly 10. In the unfortunate event that the door of the next OR is opened, then some trace of the surgical smoke emitted by the OR upstream, can flow inside the next OR down the hall. And while the level of exposure to surgical smoke would be insignificant in this second OR, it is clear that the standard positive pressure established in these ORs cannot guarantee those airborne particles do not propagate from one OR to another. Over a period of several months, this rare event might be capable of propagating an airborne disease. In fact, the frequency of door openings of each OR can be very high that the probability of propagating airborne diseases and contaminating other ORs seems inevitable. Next, to systematically assess long-term exposure, the result obtained by coupling the air quality model with an Agent-Based Model (ABM) of the surgical flow will be reported on. Notably, while exposure to surgical smoke in the OR is relatively intense in a short period of time and then vanishes, the pollutant is stagnant in the hallway for a much longer period of time and therefore contributes to long-term exposure.

A method to construct a surgical-suite-specific model of the transport-diffusion of airborne particles can quickly be calibrated with cost-effective wireless particle counters. Coupling this indoor quality model to the previous multi-scale model of surgical flow allows quantification of surgical smoke exposure across long periods of time and provides a rationale for recommendations. As a matter of fact, the ABM of surgical workflow allows insight into the human behavior factor, which can be included in the analysis. This work may expose rare events, such as contamination from one OR to another, which when accumulated over the months becomes a tangible risk. This study has potential because it can run for long periods of time and can address the complexity of hundreds of staff's spatiotemporal behaviors in a large OR suite.

The CFD model requires detailed geometric and boundary conditions to be reliable; the k-e model is an approximation that has its own limit as well. Running a CFD model is a tedious process both in setting up the mesh and simulation parameters, as well as in terms of central processing unit (CPU) time required. CFD was used here only to test the components of a hybrid stochastic compartment model that incorporates the mechanism of diffusion-transport of airborne particles at the surgical suite scale over a one-year period. A coarse statistical model was used for the source of surgical smoke. the actual generation of surgical smoke depends on the surgery team, type of procedure, and many more parameters. However, the capability to non-invasively monitor such parameters using appropriate sensors via the cyber-physical system is available. The hybrid Partial Differential Equation (PDE) compartment model provides a first-order approximation of average exposure at the room scale. The delay in the transmission conditions between the OR and the hall in Equations (4) and (5) is not essential to reproduce the result on daily exposure to smoke. In the meantime, the uncertainty of the HVAC input/output provides a much larger error. Furthermore, deposition of particles on the OR's surfaces was not taken into account. The deposition of UFP may be expected to be negligible. A low accuracy model that carries an error of the order of 20% could, however, be conclusive for this study.

As a conclusion, it is particularly important to recognize the impact of door design and human behavior when considering hazardous airborne particles spreading throughout a surgical suite. OR doors constantly leak air depending on the difference of pressure with the outside hall, and they contribute to the transport of particles throughout the surgical suite. The door opening effect depends on the motion of the door and also the difference of temperature between the OR and the hallway. Some of these negative impacts can be controlled by a better design of the door and of the temperature control in order to work with a more cost-effective HVAC design. The benefit of positive pressure in the OR is still canceled by door openings, inducing possible back-flow and contamination from the hallway, especially when the door stays open for several minutes. Therefore, efficient movements by personnel may improve indoor air quality and should be quantified. The architectural design of the OR suite should optimize the circulation of staff and patient movement activities.

In the current study, quantification of surgical smoke concentration in the hallway, the duration of exposure along the year, and the mechanism of propagation of hazardous airborne particles from one OR to another was feasible. On the practical side, an automatic sliding OR door seems to be a better solution over a traditional door's rotation that acts as a pump. The analysis can also be extended to address the problem of the optimum placement of UV lights in the hallway to improve air quality in an efficient and controlled way. Finally, the importance of AORN's guideline in the use of a vacuum system during surgery needs to be reinforced at a time when elective surgery may involve asymptomatic COVID-19 patients.

### PREFERRED EMBODIMENTS

A detailed description of the preferred embodiments of the invention is disclosed and described below. Yet, each and every possible feature, within the limits of the specification, are not disclosed as various permutations are postulated to be in the purview and contemplation of those having skill in the art. It is therefore possible for those having skill in the art to practice the disclosed invention while observing that certain placement and spatial arrangements are relative and capable of being arranged and rearranged at various points about the present invention that nonetheless accomplishes the correction of one or more of the infirmities as outlined and discussed above. Patently, the size and shape of certain features may be expanded or narrowed to accommodate each individual space and system requirement.

Inventors have set forth the best mode or modes contemplated of carrying out the invention known to inventor such to enable a person skilled in the art to practice the present invention, the preferred embodiments are, however, not intended to be limiting, but, on the contrary, are included in a non-limiting sense apt to alterations and modifications within the scope and spirit of the disclosure and appended claims.

Equally, it should be observed that the present invention can be understood, in terms of both structure and function, from the accompanying disclosure taken in context with the associated drawings. And whereas the present invention and method of use are capable of several different embodiments, which can be arranged and rearranged into several configurations, which allows for mixing and matching of features and components, each may exhibit accompanying interchangeable functionalities, which may be space and requirement specific, without departing from the scope and spirit of the present application as shown and described.

### Description of the Cyberinfrastructure

As detailed in FIG. 1, inventors' system architecture resides on-site 300 (which may be accomplished equally off-site) and within a clinic or hospital and is, preferably, behind the clinic's firewall 350 and given authorized access to each institution's local area network (LAN) 340. Inventors' graphical user interface and associated application (FIGS. 21-23) can be accessed via mobile device, computer tablet or stationary computer 330 and may act as an interface with which to monitor, track and access a space's air quality. FIG. 1 details the relationship between a mobile or computer access 330 connected to a hosted webserver 320 whereby a wireless connection 340 supports connections to an on-site server 315 (and accompanying database 320) which in turn is operably connected to both an air quality component 310 and an endo-acquisition component 305 for the real-time monitoring of ambient air quality.

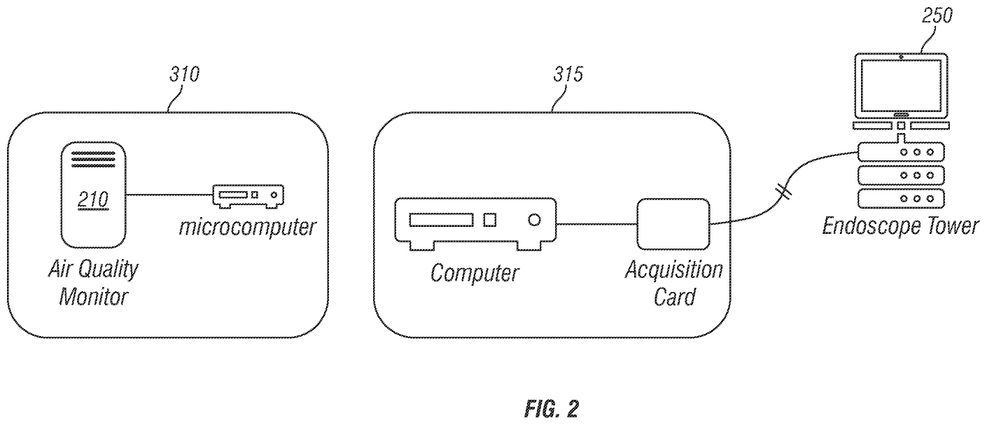

### FIG. 2: Description of the System Components

As described in FIG. 2, Inventors' system components consist of air quality sensor technology (air monitor) 210 and endoscope system technology Endo-acquisition component 305 directly plugging into the clinic's endoscopic tower 250 in the operating room (OR). Each OR consists of both components 310, 305. Extra air quality components may be placed throughout the clinic at various locations to gather further measurements as warranted and necessitated including inventors' air quality device 10 enumerated below.

### All-In-One Device

In an attempt to maximize the current infrastructure, inventors have developed a modular device which sends real-time recommendations to manage risk of airborne disease or contamination due to aerosols wherein large quantities of aerosol can possibly be generated by a procedure and expose medical staff in that procedure room to the virus load from the patient.

Integrated within the present system, the present device is designed to be a compact container that is easy to install and/or retrofit into any existing standard procedural room. Said device is instituted to monitor air particulate density in such a way that that exposure to aerosol during the length of stay in the procedural room from airborne particle can be monitored continuously to provide feedback on safety and support may be provided where an “air quality turnover protocol” is utilized such that airborne particles harbor minimum risk of propagating from one cycle of the room procedural usage to another.

A good example of the application of the present device's use is to mitigate the risk of virus infection (e.g., COVID-19) by airborne particles from one patient to another in any outpatient center that practices a large volume of short procedures such as dental care, laser surgery to improve vision, routine colonoscopy to prevent cancer and the like. The primary motivator is that the risk for any patient in a given clinic day should be as low as the first patient of the clinic day who benefits from a very low particle density air if the procedural room was properly closed at night, sterilized and the HVAC system was operating properly. This best ensures a patient's safety while in a procedural area.

Inventor's “all-in-one device” (i) measures particle density (specifically small particle density), (ii) translates that information into an autonomous support management system assisting staff in monitoring and managing room turnover, and (iii) mitigating the risk of airborne disease infection according to certain benchmarks and parameters designed for a particular facility.

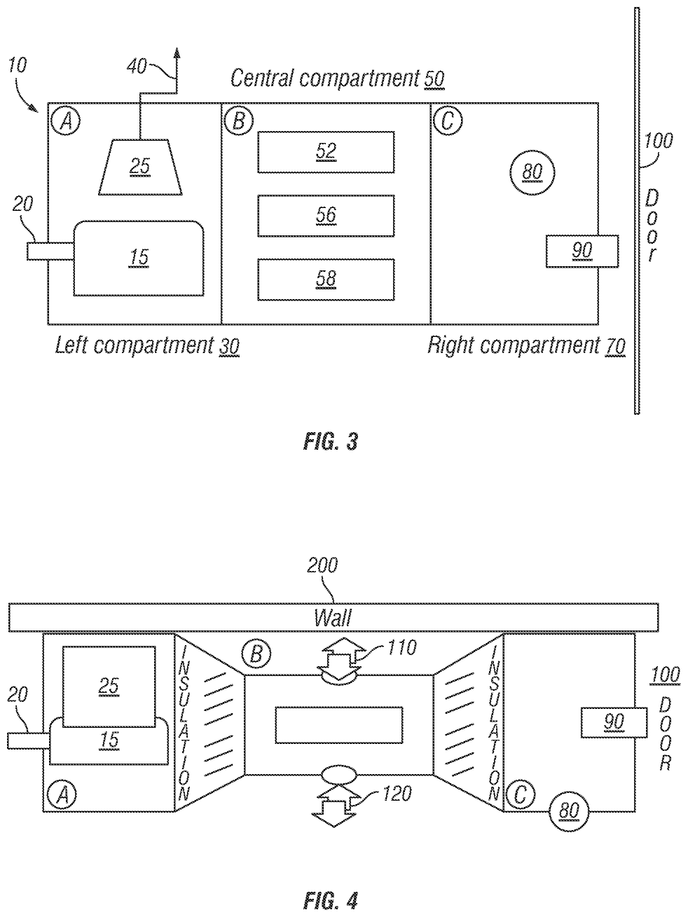

As depicted in FIG. 3, the air monitoring device 10 that is the present operable particle sensor is configured into 3 compartments: a “left” compartment 30 for computer electronics, a “center” compartment 50 for air quality sensors and a “right” compartment 70 for room state sensors which may be reversed or spatially rearranged as operation dictates. The “left” compartment 30, compartment A, contains a microcomputer 15 to process sensor data and any additional external input link of information, such as EMR time stamps, an externally residing Wi-Fi antenna 20 and a power source 25 and connectivity (ex. plug) 40 preferably with optional Uninterruptible Power Source (UPS) to ensure system integrity. The “central” compartment, compartment B, contains 1 to 3 particle sensors 52, 56, 58 (with an additional number of sensors for redundancy, verification and expansion of operations where two air quality sensors are different models and cross check each other continuously). The “right” compartment, compartment C, contains an infrared (IR) sensor 80 to detect motion (door or staff motion) and/or an ultrasonic sensor 90 (detecting door or staff movement). Representationally, a door 100 is illustrated which may as well display a mounted reflector to enhance Ultrasonic (US) distance measurement accuracy.

FIG. 4 is the previous detection device 10 of FIG. 3 (above) wherein channel 110 and channel 120 are inflow/outflow channels, interchangeably, feeding air particle sensors and gas sensor(s) within compartment B. This “butterfly shape” allows proper gas exchange to improve the quality of measurement and offer a convenient solution for calibration when needed.

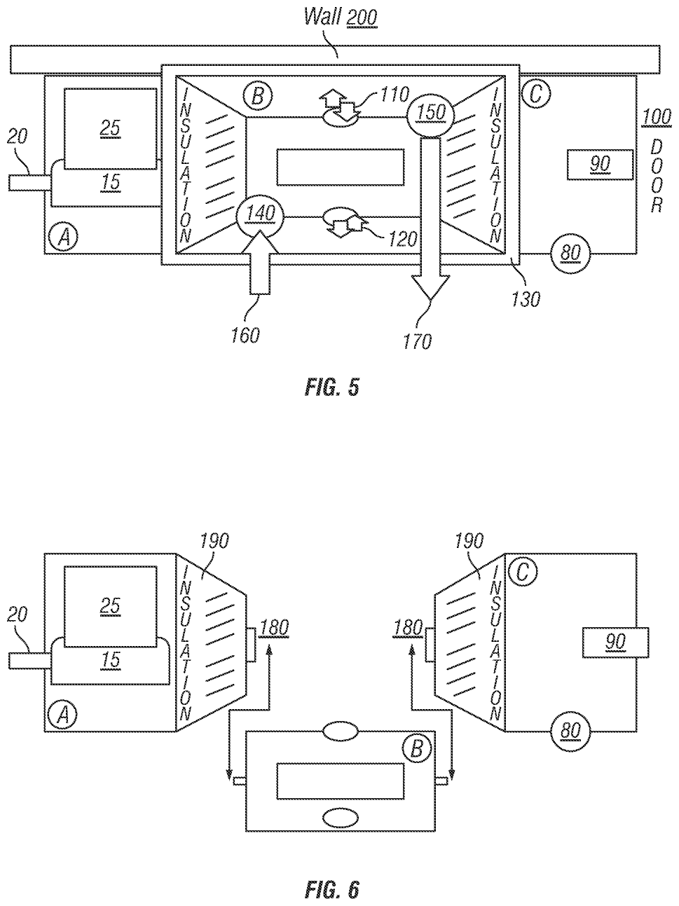

FIG. 5 is the previous detection device 10 of FIGS. 3 and 4 (above) with the addition of a frame 130 which can be temporary mounted on the device to isolate the central compartment for air quality sensors 52, 56, 58, inlet valve 140 is the inlet valve for quality control (zero) gas, outlet valve 150 acts as an exhaust vent, inlet 160 may be used to insert or mount a gas bag or syringe to input control quality gas and exit valve 170 may be used to recover control quality gas.

FIG. 6 depicts a device 10 which is reflects in FIGS. 2-5 above, but that is, in addition, modular wherein an operator can update each module separately when a new generation of microcomputer 15 or sensors (52, 56, 58) become available or replace a module if its components fail without having to replace the entire system (where each separate component may be separated and “unplugged” form the other). Of importance is insulation (via insulators 180) between components whereby each of three compartments are isolated from each other with insulators (ex. foam) to limit the potential impact of heat generated by the equipment on the accuracy of sensors and the computer electronics compartment, in particular, is equipped with a proper heat sink and fan to cool the microcomputer 15. Too, connectors 180 are present to allow for connections between compartments A, B and C.

Of special importance, the inlet compartment can be easily closed with a cover that can be clicked or snapped in place. One can use a known quantity of pure air particles to check and/or calibrate said sensors as a baseline (i.e. pure air particle calibration), as well as a known quantity of Small Particles (SPs) with known “calibrated” particle count mounted on a closed compartment to check and/or calibrate the particle and/or gas sensors with a known quantity of pure air particles allows checking the calibration of the zero state in particle counts via a gas container (e.g., bottle or spray can) allows the operator to check the calibration of the SP count and/or gas composition (provided the gas container can push a known quantity of SP in a pressured flow once the valve is open; an alternative solution is to use a standard, commercially available aerosol spray, provided that consistency and quality can be verified on the product from the vendor or via independent third parties). If the calibration is incorrect the user can replace the non-functional or dysfunctional components and plug-and-play a new air quality module or component part.

In terms of operational and computational components, the“left” compartment (Processing Unit and Wi-Fi connectivity) comprises:
a. A microcomputer, such as a Raspberry Pi
b. Wi-Fi antenna for internet capability, such as a Wi-Fi USB adapter/dongle
c. Power Supply with possibly a UPS (Uninterruptible Power Supply/Source) system with a battery to guarantee the quality of service during sudden loss of power.

In terms of air quality monitoring, the “sensor” or “central” compartment comprises Air quality (AQ) sensors, comprising:
a. Gas sensor for volatile organic compounds (VOC) (optional), for example bme680 temp/humid/gas sensor; https://www.adafruit.com/product/3660
b. A first Optical laser particle count such as Plantower® PMS7003 G7 PM2.5 Sensor Laser Particle Sensor Detector Air Quality Tester
c. A second (or third) Optical Laser particle count for redundancy

Room state sensors can be without limitation, comprising:
a. Ultrasonic (US) sensor to detect door openings wherein the device is mounted close to the door frame in order to detect door openings and/or closings as an obstacle
Note: When the door is a sliding door, inventors mount a reflector that is a small plate perpendicular to the door plane and face the US probe.
b. Infrared (IR) sensor to detect motion in procedure room
c. Light sensor (optional) to detect when the light is on or off. Typically, the light should be off when the room is not used for a long period of time. In various procedures, such as minimally invasive surgery, the light is dim to allow a better view of display by operators. This allows another data point on room state.
d. Noise sensor (optional) where noise is also a channel of information to arrive at room state.
e. Antenna to capture presence of Bluetooth low energy (BLE) or Radio-frequency identification (RFID) tags/devices (yet another possibility for room monitoring)

In one preferred embodiment, two particle sensors check SPC on a continuous basis and compute the air particle count once every minute. Yet, this time period is merely exemplary and may occur in a sub-minute timeframe or may occur in periods longer than one minute as conditions or requirements necessitate. As shown in FIGS. 5 and 6, sensors provide a signal that is strongly correlated to room state in surgery, as well as in gastroenterology procedures. Similar correlation can be established between procedures and AQ sensor signals for other applications, such as dentistry, laser intervention in ophthalmology and the like. Any disparity between these two sensors, or potentially among a plurality of sensors, beyond a preset r-tolerance number, will create an alarm to replace the central bloc of the device with a new set of pre-qualified and pre-calibrated AQ sensors.

In another embodiment, the gas sensor can be of any type and is optional wherein the reliability of low-cost gas sensors is poor and generally requires 3 months of monitoring—see [1,3]. Calibration of the gas sensor can be done in a similar way as the calibration of our dual air quality sensor thanks to the butterfly design with quick, closed compartment mounting.

In yet another embodiment, the digital sensors such as IR and US check the presence of staff and patients in the room and the door openings and closings. These two channels of information provide some redundancy and context thereby assuring proper measurement and verifiability of results. Sound and light levels and activity within a room can be monitored by additional sensors, as warranted, which provide additional means by which to procure activity data. The Raspberry Pi collects all 4 to 6 sensor outputs. The code is in Python for scalability, efficiency, and portability purposes.

The system has standard real-time locating system (RTLS) capability to identify if specific items enter or leave the room providing detection for RTLS tags associated to patients, medical equipment (e.g., beds), medical staff or customers. In the present iteration, inventors use in standard mode Bluetooth low energy (BLE) technology and off the shelf BLE tag assemblies as to monitor and manage patients, medical equipment, medical personnel and cleaning staff. Equivalently, RFID tags may be utilized to the same effect and may be preferable in some situations.⁴³

If inventors use the present device and system in conjunction with the software generation (Gen1.5) of a software platform for GI procedures, the present technology can be utilized to verify that the certain equipment (e.g. an endoscope tower or cleaning cart) has been correctly identified and spatially located via an attached tag (i.e. BLE or RFID tag), for example, wherein the present device can detect the inventory and location of essential pieces of equipment thus providing key data informing the procedure room state (ex. whether in use, for what type of procedural use and in what stage of procedural use). And, likewise, the present device can use similar identifying tags to inform the system as to a piece of medical equipment's operability and reliability by reading a particular input received and output transmitted by said a tag. These tags can actively or passively (in the case of RFID) transfer information confirming proper placement and functioning of medical equipment within a procedure room or, alternatively, send an alarm alerting operators of this equipments improper placement, illegitimate use or functional deficiencies.

In another embodiment, a first interface to the system can be comprised of a “three-traffic light assembly” positioned outside of an operating room and connected to the present device via a wire (or wirelessly) wherein, for example, a red light signifies that a procedure room is occupied (has not been properly sanitized and/or should not be entered), an orange light signifying that the AQ turnover process is ongoing and in process, and a green light signals that the room is available for the next patient procedure. A second interface can be a mobile phone application, desktop or tablet application for a single user that provides essentially the same or similar information, in the same or different formats, as well as time estimates for procedures and pre and post procedure events. And a possible third interface, which can be a website that delivers statistic, global info, automatic report editing at specific times, which is designed primarily for administrative purposes (e.g., the manager of the facility) for scheduling and maintenance of workflow. Further, FIGS. 1, 2, 21-23 provides an example of our implementation for an outpatient GI center's configuration and interfaces. This implementation is compatible with any device such as a computer display, tablet or cell phone.

The system may communicate with the user by delivering text messages using a texting service, for example Twilio [https://ahoy.twilio.com/], or through a graphical user interface (as above) that is available for access on a cell phone, a tablet, or computer display as detailed below by:
(i) Providing information on room readiness for next patient, medical personnel or team to enter a surgery room and/or availability of a procedure based on a minimum airborne particle threshold (e.g., an AQI of 50 or less correlated to number of 2.5 microns particles per meter cubed);
(ii) providing information on when cleaning is complete, room is empty air has been recycled by the HVAC to an AQI of 50 or less for next patient/procedure;
(iii) providing text messages to alert operators to check calibration regularly with provided containers (bags/syringes as above);
(iv) providing a quick method to spot-check calibration of air quality sensors manually, date/time of last calibration or instant AQI;
(v) providing information of patient presence, staff presence, both or neither in the procedure room based on RTLS tag to compute exposure during the room activity;
(vi) providing presence and spatial information of surgical equipment in the room (or absent) based on RTLS tag for inventory or workflow purposes;
(vii) sending an alarm via an interface (through text message or on cell application) if door opens during turnover air quality process (when in process of air renewal),
(viii) computing actual room occupancy versus optimum or capacity room occupancy based on optimum AQ turnover;
(ix) computing small particle amount and thresholds in procedure room during processes;
(x) computing small particle thresholds based on benchmarking and/or base line of the facility with closed doors and no activity for extended period of time;
(xi) comparing room performances to other rooms within a facility, other facilities and theoretical or historical rooms or facilities and providing recommendations to staff and managers;
(xii) interconnecting with real-time procedure monitoring signals, if available, to anticipate turnover, delays and other workflow milestones and criteria;
(xiii) interconnecting with electronic medical record and scheduling systems, if available, to anticipate and manage delays, to better organize turnover and to streamline procedures while observing best AQ practices.

### Particle Count During a Clinical Day in a Procedural Room (FIGS. 7 & 8)

FIG. 7 shows procedure state (top graph) and particle counts (bottom graph), on the vertical axis, respectively, in a procedural room during a clinical day in an outpatient center where time is measured on the horizontal axis. For simplicity, inventors selected a simplified, and therefore light, activity day in a procedural room for a representative procedure set with only three patient procedures. The procedure state (FIG. 7 top) provides the procedure room state wherein procedure times start shortly before 8a, shortly after 9a and shortly before 11a and are marked as a colonoscopy. The first patient underwent an EGD from 7:40 am to 8:00 am, the second patient received a colonoscopy starting approximately 9:00 am, and the third patient received a colonoscopy around 11:00 am. Noticeably, while the start and stop times are relatively short intervals, the turnover time between procedures is extremely large, i.e. about 1 hour for the first interval between the first and second patient procedures, and roughly 1.5 hours for the second interval between the second and third patient procedures. This turnover time is very unusual since this time in this clinic prior to the COVID19 pandemic, was approximately 10 minutes on average. Turning to FIG. 8, the 3 procedures' duration are similar to procedure durations before COVID19, i.e. about 10 minutes for an EGD and 20 minutes for a colonoscopy. The step function in the top part of FIG. 8 shows the endoscope state of operation (defined below). Here the first procedure is an EGD and the second and third are a coloscopy. The procedure time is defined as the time period when the flexible endoscope is inside the patient. Initially, the step functions stay at zero value until 7:00 am, which corresponds to the endoscopic tower not in use or not powered up. When the step function is at 1, the flexible endoscope is not plugged into the endoscope tower but the endoscope tower has been powered on. When its value is 2, the flexible endoscope has been plugged in but is not in use for the procedure. When its value is 3, the procedure is occurring (i.e., where the endoscope tower is on, the endoscope is ‘plugged in’ and the flexible endoscope is in use for the diagnostics and/or therapy). The scope state follows this cycle [1 2 3, 1 2 3 . . . ] until the last procedure is finished.

Markedly, the flexible endoscope was ready to use long before the patient procedure started for patient 1 and 3, but not for patient 2 (defined as a long ‘2’ interval for patient 1 and 3, but not for patient 2). Of note, in a standard workflow, the flexible endoscope is brought to the procedural room about 5-10 minutes before the procedure. This was the case for procedure 2. It is rather uncommon that the flexible endoscope arrives more than half an hour before the procedure (inducing a risk of contamination). The typical clinical day ends around 4:00 μm and the endoscope tower is eventually powered down, so the scope state will return to 0.

The bottom graphs of FIG. 7 and FIG. 8 track particles of small size in the range from 0.5 to 2.5 microns, which are the focus of the present invention and primary discussed vehicles of virus-sized of biological material. The less pronounced (lower) peaks in FIG. 7 track particles of larger size (greater than 2.5 microns in diameter) and the more pronounced (upper) peaks track small particles of less than 2.5 microns in diameter. For the aforementioned reasons, inventors focused on these small particles (less than 2.5 microns in diameter) for their ability to stay aloft and airborne longer than large particles, their ability to penetrate further into the respiratory tract and pose the greatest “physical” source of transmission and potential contamination. As provided above, a Small Particle Count (SPC) below 50 AQI is considered to be “excellent” air quality according to the EPA (and subsequently per the Dylos manufacturer tested requirements) whereby those small particles with the greatest capacity to transmit a viral infection are reflected in this particle size range (0.5-to-2.5-micron diameter per m³) wherein particle count is inversely related to air quality (i.e., as the particle count number goes up, air quality degrades). In all of the present measurements, inventors observed that an SPC of less than 50 (AQI), when the procedural room is empty with the door closed, represented the real-time measurement of efficiency of the HVAC system at filtering out particles to an acceptable level as designed and expected within a much smaller interval than previously theoretically calculated. Inventors thus consider this level of particle count as the baseline for insuring an acceptably low levels of small particles (i.e., low SPC).

A few observations can be taken from FIGS. 7 & 8. As mentioned above the SPC (as correlated to AQI) is far below 50 (SPC/AQI) until 6:30 am, which shows little to no small particles and/or respiratory droplets containing particulate matter and, indirectly, that the procedure room was ‘sealed’ and the door of the procedural room was closed until 6:30 am. Upon the entrance of medical staff, and/or their entrance and egress, or the door maintaining a transitory or primarily open state, the small particle counts correspondingly begin to rise due to entrance of exterior particulates. Additionally, as can be identified on the upper chart of FIG. 8, the endoscopic state of 2 indicates that the flexible endoscope was connected but not yet activated in the procedural room around 7:00 am, and so inventors observed a small spike (in lower FIG. 8) in the SPC/AQI corresponding to this time period. As a visual reference on the particle count graphic (FIGS. 7 and 8 bottom charts) the procedure beginning, as indicated by a vertically descending segment on FIG. 7 and FIG. 8, it may be observed that air quality changes rapidly with the initiation of the procedure and, systematically, a peak in the particle count approximated by the procedure beginning (i.e., when the flexible endoscope is introduced into the patient's esophagus). In contrast, the particle count immediately before that time is demonstrably less, even while both the patient and the team occupy the room for several minutes preceding the procedure and the door of the procedural room is in a closed state. This peak is not relative to, and cannot be attributable to, medical staff already inside the room but rather is commensurate with those aerosols resulting from fluids emanating from the patient and the procedure itself. This observation is consistent with other findings that both EGD and colonoscopy are generators of significant aerosolized particles including pathogenic dispersants.⁴⁴

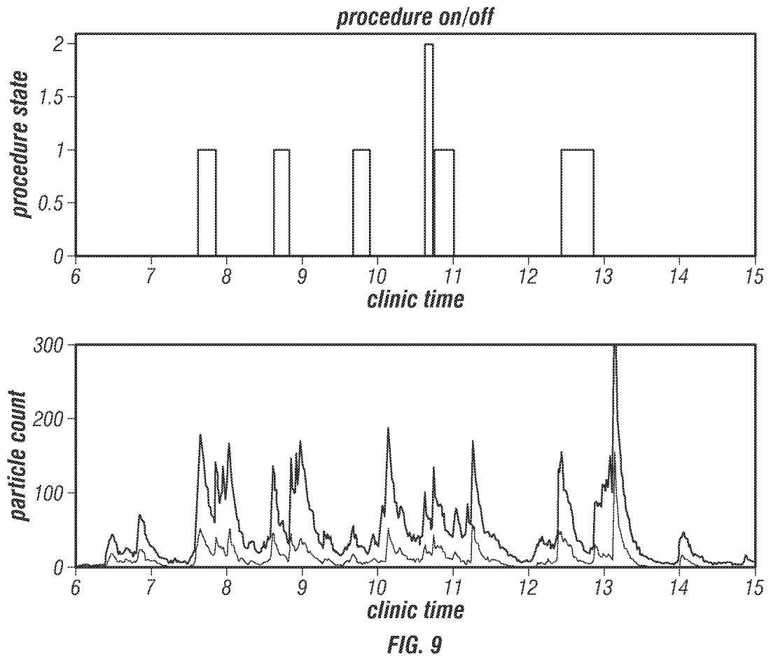

### Example of Particle Count During a Clinical Day (FIGS. 9 & 10)

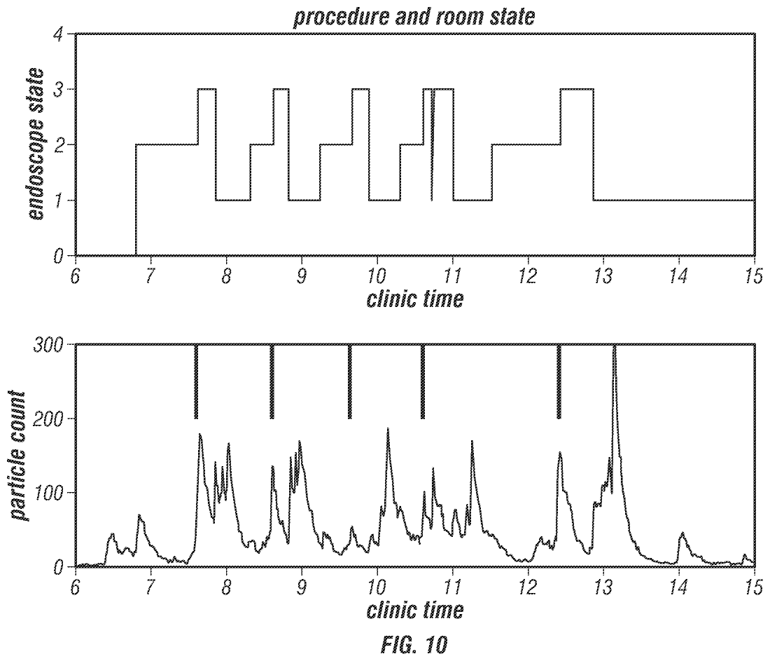

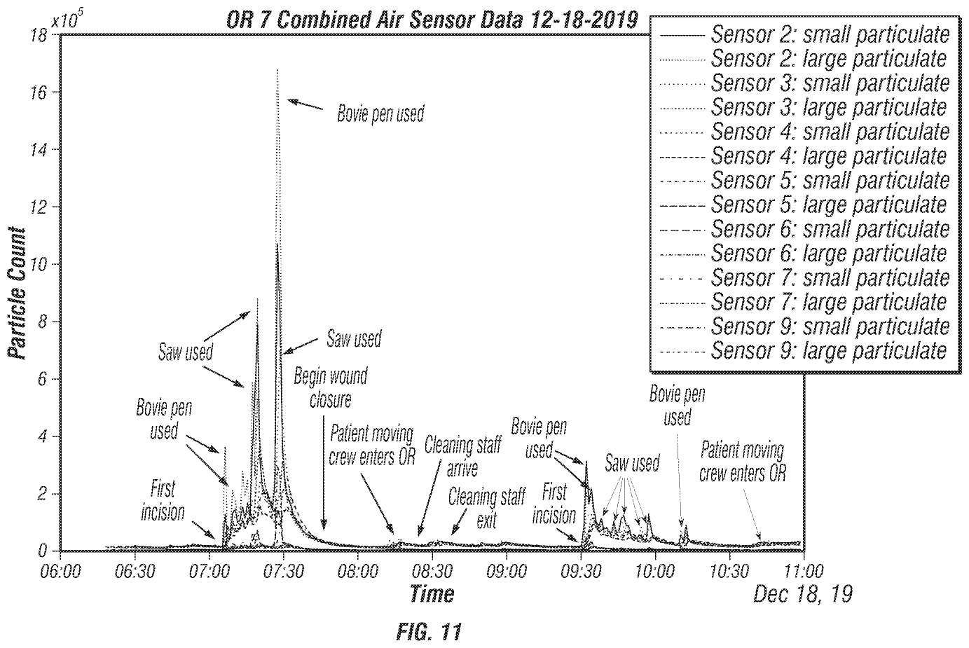

The same observations are documented and confirmed by similar patterns in different procedural rooms conducting the same GI procedures within the same facility having six procedures occur as opposed to three, as shown in FIGS. 9 and 10. Of note these 6 procedures were conducted on five patients, where the fourth patient received two procedures: an EGD immediately followed by a colonoscopy where the less pronounced (lower) peaks in FIG. 9 track particles of larger size (greater than 2.5 microns in diameter) and the more pronounced (upper) peaks track small particles of less than 2.5 microns in diameter (the subject of the present invention). Once again regarding this second data set, the duration of the procedures themselves were relatively standard and expectantly short, but the turnover time between procedures was measurably larger compared to traditional pre-COVID workflow statistics and measurements.

Another remarkable feature of the SPC (relative AQI) measured is that it yields the time at which the janitorial team arrives in the procedural room in order to clean surfaces: this process generates significant amounts of aerosol with the evaporation of disinfectant product. It can even be seen that at the end of the procedure day (between 11 and 12 on FIGS. 7 and 8 bottom and after 13 on FIGS. 9 and 10 bottom corresponding roughly to clinic times 1130 and 0110, respectively), procedure room cleaning is accomplished more rigorously in both procedural rooms which generates, by far, the largest peak of measurable particle counts (both (a) below 2.5 micron diameter and (b) above 2.5 micron diameter but below 10 microns).

Further, FIG. 10 represents the endoscopic state (similar to FIG. 8) on the top graph and particle count on the bottom graph, vertically, on the X axis, wherein both graphs are charted over time, horizontally, on the Y axis. Additionally, procedure start times are designated as linear, descending lines vertically. Each evidenced maximum peak corresponding to periods post procedure initiation as a result of GI procedure generated aerosols.

### Combined Air Sensor Data (OR 7)

FIG. 11 shows a granular, 9 sensor depiction of various surgical implements in a single OR setting including use of a Bovie pen, scapple, saw and movements of patient and cleaning crew. Additionally, inventors were able to, through various sensors (2-9), each sensing both small and large particles, differentiate particle generation due to surgical sources (i.e., (1) between 7:00 am and 8:00 am and (2) between 9:30 am and 10:30 am) from those particle generated due to patient moving (between 8:00 am and 8:30 am and 10:30 am and 11:00 am) and those particles generated by the cleaning staff (through movement and cleaning activities) which occurred shortly before 8:30 am and after 8:30 am on this representative graph.

SPC in the First 5 Minutes of a Colonoscopy Vs. The SPC 5 Minutes Prior to the Procedure

In FIG. 12, as represented by a scatter diagram, represents the ratio of the mean SPC during the first 5 minutes of a colonoscopy divided by the mean SPC during the 5 minutes prior to the colonoscopy procedure wherein there exists an appreciable and statically significant skew of aerosolized particles toward the start of the procedure (Y-axis) versus prior to surgery (X-axis). Typically, during this time window, the patient is already inside the procedural room along with the medical team, composed of the gastroenterologist, registered nurses, anesthesiologist, and a one or more technicians, among other potential participants, conducting the endoscopy. Inventors used this time window as a basis and baseline to rate the particle count generated from the procedure itself, wherein the door is closed and the number of people who are in the procedural room is constant. Inventors observed that in the vast majority of the cases this ratio is concentrated above the y-x line (bisecting the upper x-y domain and lower x-y domain) and demonstrably concentrated closer to initiation of the colonoscopy procedure.

### SPC in the First 3 Minutes of an EGD Versus the SPC in the 5 Minutes Prior to the Procedure

As expected, this result is replicated in terms of EGD procedures and may be relied upon for appropriate inferences with regard to the physical properties of aerosolized respiratory droplets and small particles in both colonoscopy and EGD procedures where aerosolized droplet production is skewed toward time closest to each procedure.

### Distribution of Indicator of Aerosol Generation for a Colonoscopy During the First 5 Minutes of the Procedure

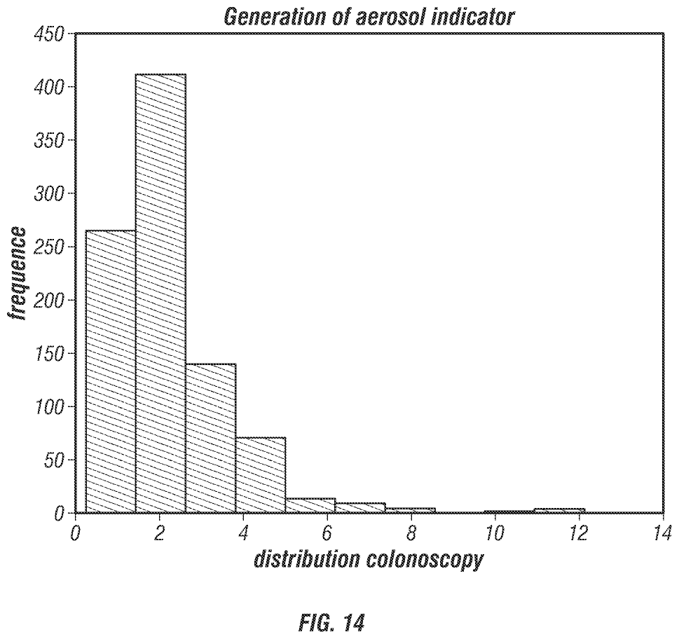

FIG. 14 gives the histogram of the ratio of the mean SPC during the first 5 minutes of a colonoscopy divided by the mean SPC during the 5 minutes prior to the colonoscopy procedure. showing a biased distribution with 2 minutes as its maximum. According to our interpretation, these figures show that colonoscopy procedures generate a significant level of aerosol.

### Distribution of Indicator of Aerosol Generation for an EGD During the First 3 Minutes of the Procedure

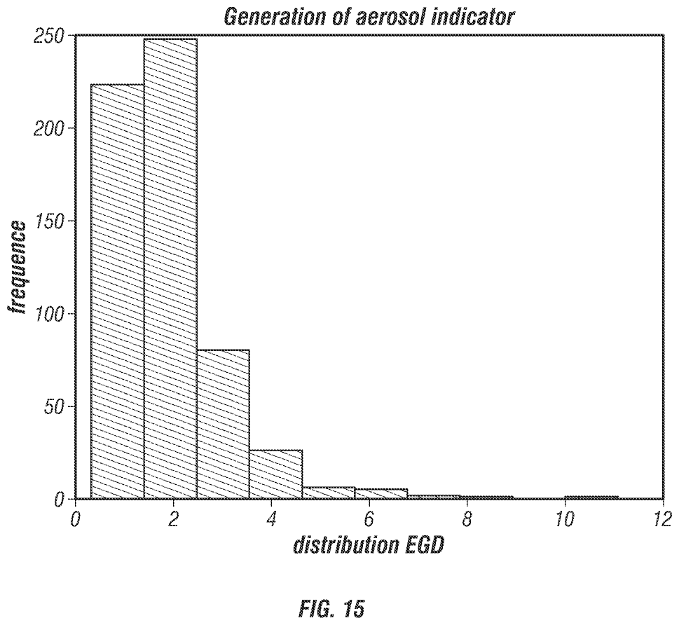

FIG. 15 gives the histogram of the ratio of the mean SPC during the first 3 minutes of a EGD divided by the mean SPC during the 5 minutes prior to the colonoscopy procedure. This Figure shows a biased distribution with 2 minutes as its maximum (with a close peak at 1 minute). According to our interpretation, these figures show that EGD procedures generate a significant level of aerosol.

Distribution of Indicator of Aerosol Generation for a Colonoscopy after the Initial Phase of the Procedure

In this FIG. 16 inventors show a histogram that gives the distribution of the indicator of aerosol generation for each colonoscopy after the initial phase of the procedure. Inventors observed a log normal distribution where about 40% of the procedures have an indicator above one showing that procedures still generate significant level of SPC during that phase.

Distribution of Indicator of Aerosol Generation for an EGD after the Initial Phase of the Procedure

In this FIG. 17 inventors show a histogram that gives the distribution of the indicator of aerosol generation for each EGD after the initial phase of the procedure. Inventors observed a log normal distribution; about 40% of the procedures have an indicator above one, which shows that procedures still generate significant level of SPC during that phase.

Relaxation Factor Lambda of Particle Count Decays Approx. Exp (-Lambda t) for Small Particles (Black) and Respectively for Large Particles (Red)

FIG. 18 shows the mean and standard variation of the lambda value obtained from each fitting in each procedural room. Inventors present the same calculation but for the large particle count using the same time interval. The smaller the relaxation factor, denoted lambda, the longer the time period for the particle count to decay to a given threshold level. The relaxation factor for small particle is smaller than the relaxation factor for large particle no matter the procedural room. As expected, the large particles are eliminated faster than small particles, which confirms the relative higher risk for SPC than Large Particle Count (LPC).

### Air Quality Turnover Time Measured in Clinical Conditions

Air quality turnover in FIG. 19 varies significantly across procedures and procedure rooms wherein, in a given room, the air quality (post cleaning) varies based on multiple factors such as previous patient conditions, surgical teams, duration of cleaning and so on.

Overall, the air quality turnover time varies on average from 7 to 15 minutes depending on the procedural room and several multivariant factors. Although, this variability is not a determinative factor in the actual determination of acceptable particle count (SPC) reflected in an AQI below 50 which is, in turn, considerably faster than the theoretical prediction, based on predictive vales in HVAC systems, represented in FIG. 20.

Turnover Time Computed from the HVAC Formula Provided by the CDC Website

As provided above, FIG. 20 depicts a representational theoretical refractory time period of HVAC systems in procedure rooms 1 through 7, based on CDC provided calculations, ranging from 14 minutes (procedure room 5) to 35 minutes (procedure rooms 1, 2 and 7).

### Graphic User Interface

FIG. 21 illustrates a GUI dashboard wherein (1) number and type of GUI views is configurable, (2) number of spaces (squares) for procedure rooms is configurable, (3) number of horizontal timelines is adjustable and “stackable”, (4) colors are configurable and interchangeable and (e.g., blue corresponds to upper Gi and red corresponds to lower GI) (5) procedure type is mutable (changing colors, shapes or configuration from horizontal to vertical) to accommodate user preference.

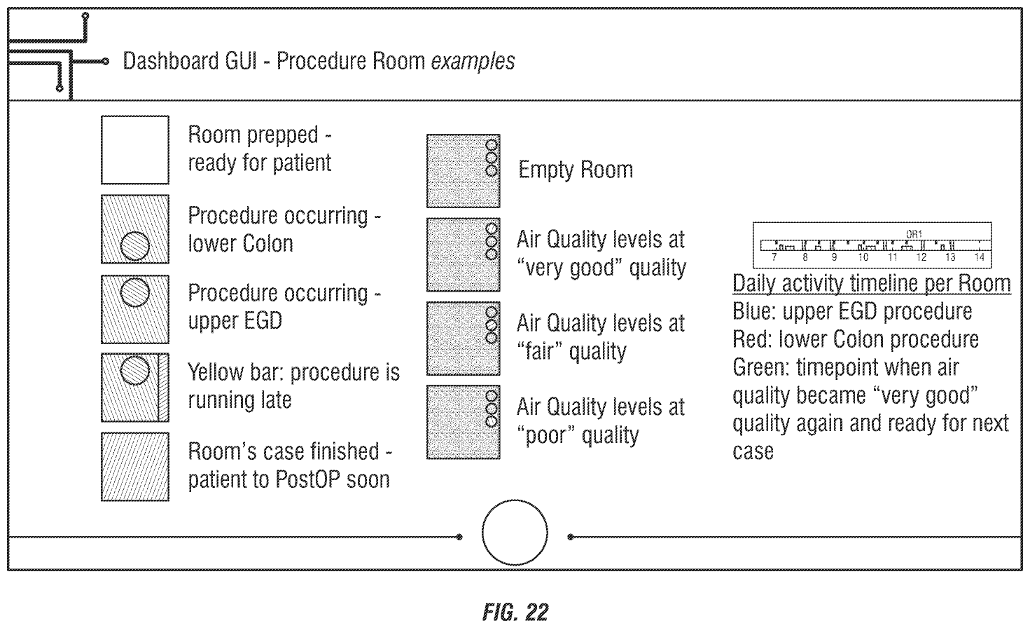

FIG. 22 give various visual representations including air quality levels (very good, good, fair and poor), stages of readiness (prepped or staged), occupancy of room (ex. occupied, empty, surgery complete but patient remains in room), status of room (ex. clean, undergoing cleaning, clear, open, on time, running behind), type of procedure (lower colon, upper EGD or both), status of patient, or a combination thereof, wherein indicators may occupy a spatial relationship within a designated room icon (i.e., circle in upper box signifying upper GI, green designating AQI of 50 or below, red designating lower colon and the like).

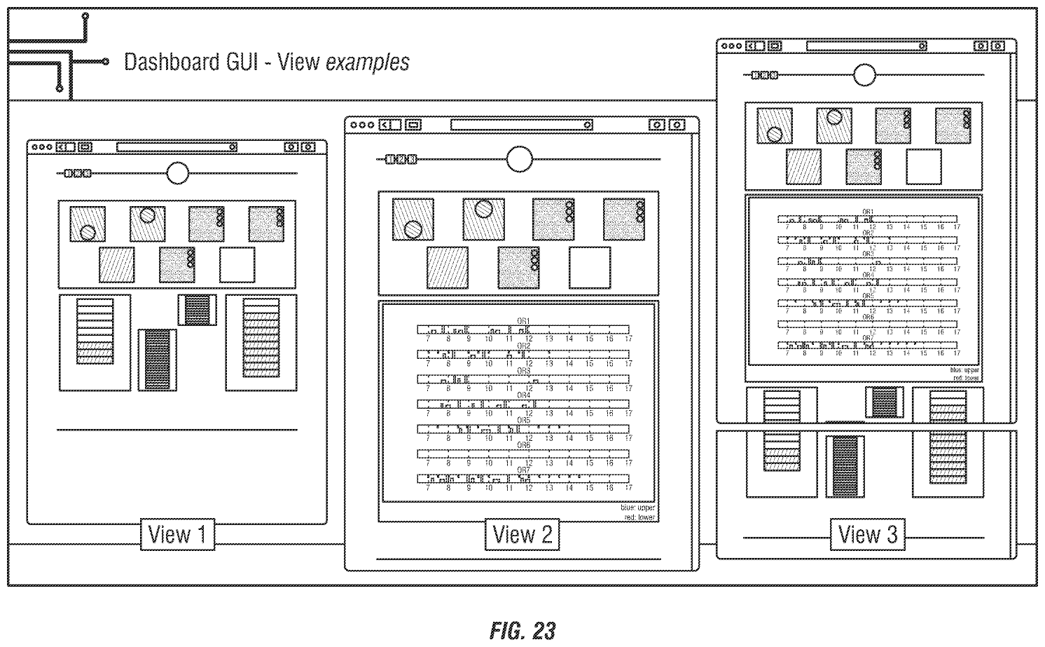

FIG. 23 provides for various and variable views (as signified as 1, 2, 3 in FIG. 21 top left) based on the user's preferences whereby designated perimeters can be configured to provide granular views down to a particular space and more holistic views of all spaces. Representationally, user may select a number corresponding to a particular view, user may “swipe through” screens for desired selection, “shrink” and “expand” said screens, toggle between and among screens or a combination thereof.

It is to be understood that the disclosed embodiments are merely illustrative and that forms and designs of the apparatuses, systems and methods shown and described herein are to be taken as the presently best known means of accomplishing the present invention. Elements and materials may be substituted for those illustrated and herein described, parts and processes may be rearranged, and certain features of the apparatuses, systems and methods may be utilized independently, all of which would be apparent to one having skill in the art having the benefit of this present disclosure. Changes, amendments and modifications may be made in the elements described herein without departing from the spirit and scope of the invention as described in the following claims.
¹ WHO Coronavirus (COVID-19) Dashboard: https://covid19.who.int/
² Al-Jabir A, Kerwan A, Nicola M, et al. Impact of the Coronavirus (COVID-19) pandemic on surgical practice—Part 1. Int J Surg. 2020; 79:168-179. doi:10.1016/j.ijsu.2020.05.022
³ Al-Jabir A, Kerwan A, Nicola M, Alsafi Z, Khan M, Sohrabi C. O'Neill N, Iosifidis C, Griffin M, Mathew G, Agha R. Impact of the Coronavirus (COVID-19) pandemic on surgical practice—Part 2 (surgical prioritisation). Int J Surg. 2020 July; 79:233-248. doi: 10.1016/j.ijsu.2020.05.002. Epub 2020 May 12. PMID: 32413502; PMCID: PMC7217115
⁴ Anne F. Peery. Seth D. Crockett, Caitlin C. Murphy, Joseph Galanko, Todd H. Baron, Robert S. Sandler et al. Burden and Cost of Gastrointestinal, Liver, and Pancreatic Diseases in the United States: Update 2018, Gastroenterology AGA, SPECIAL ARTICLE|VOLUME 156, ISSUE 1, P 254-272.E11, Jan. 1, 2019, Published: Oct. 10, 2018DOI:https://doi.org/10.1053/j.gastro.2018.08.063
⁵ Research i. An Astounding 19 Million Colonoscopies are Performed Annually in The United States. (2018). Available online at: https://idataresearch.com/an-astounding-19-million-colonoscopies-are-performed-annually-in-the-united-states/ (accessed Aug. 8, 2018).
⁶ Peter Aaby, Jette Bukh. Jorgen Leerhey, Ida Maria Lisse, Carl H. Mordhorst, Ib Rode Pedersen, Vaccinated Children Get Milder Measles Infection: A Community Study from Guinea-Bissau, The Journal of Infectious Diseases, Volume 154. Issue 5. November 1986, Pages 858-863, https://doi.org/10.1093/infdis/154.5.858
⁷ Zhang W, Cheng W, Luo L, Ma Y, Xu C, Qin P, Zhang Z. Secondary transmission of coronavirus disease from presymptomatic persons, China. Emerging infectious diseases. 2020 August; 26(8):1924.
⁸ Pollock, A M, Lancaster. J. Asymptomatic transmission of covid-19. BMJ 2020; 371:m4851. https://doi.org./10.1136/bmj.m4851 (Published 21 Dec. 2020)
⁹ Yanes-Lane, Mercedes, Nicholas Winters, Federica Fregonese, Mayara Bastos, Sara Periman-Arrow, Jonathon R. Campbell, and Dick Menzics. “Proportion of asymptomatic infection among COVID-19 positive persons and their transmission potential: A systematic review and meta-analysis.” PloS one 15, no. 11 (2020): e0241536.
¹⁰ Fennelly, Kevin P. Particle sizes of infectious aerosols: implications for infection control. Lancet Respir Med 2020. Accessed Aug. 14, 2020 at https://doi.org/10.1016/ S2213-2600(20)303234
¹¹ Lotfi, Melika et al. “COVID-19: Transmission, prevention, and potential therapeutic opportunities.” Clinica chimica acta; international journal of clinical chemistry vol. 508 (2020): 254-266. doi:10.1016/j.cca.2020.05.044
¹² Siegel J D, Rhinehart E, Jackson M, Chiarello L, Health Care Infection Control Practices Advisory Committee, CDC 2007 Guideline for Isolation Precautions: Preventing Transmission of Infectious Agents in Healthcare Settings. Am J Infect Control 2007; 35: S65-164
¹³ Setti, Leonardo and Passarini, Fabrizio and Gennaro, Gianluigi De and Barbieri, Pierluigi and Perrone, Maria Grazia and Borelli, Massimo and Palnusani, Jolanda and Gilio, Alessia Di and Piscitelli, Prisco and Miani, Alessandro, Airborne Transmission Route of COVID-19: Why 2 Mleters/6 Feet of Inter-Personal Distance Could Not Be Enough, 2020. Multidisciplinary Digital Publishing Institute.
¹⁴ Brown J H, Cook K M, Ney F G, Hatch T. Influence of particle size upon the retention of particulate matter in the human lung. Am J Public Health Nations Health 1950, April; 40(4):450-80. doi: 10.2105/ajph.40.4.450. PMID: 18017198; PMCID: PMC1528473.
¹⁵ Valentyn Stadnytskyi, Christina E. Bax, Adriaan Bax, Philip Anfinrud. The airborne lifetime of small speech droplets and their potential importance in SARS-CoV-2 transmission. Proceedings of the National Academy of Sciences June 2020. 117 (22) 11875-11877; DOI: 10.1073/pnas.2006874117
¹⁶ Tran K, Cimon K, Severn M, Pessoa-Silva C L, Conly J. Aerosol generating procedures and risk of transmission of acute respiratory infections to healthcare workers: a systematic review. Plos one. 2012; 7(4):e35797. DOI: 10.1371/journal.pone.0035797. PMID: 22563403: PMCID: PMC3338532.
¹⁷ Angelos G, Dockter A G, Gachabayov M, Latifr R, Bergamaschi R. Emergency Colorectal Surgery in a COVID-19 Pandemic Epicenter. Surgical Technology International. 2020 May: 36:18-21. PMID: 32289868.
¹⁸ Barrett, W. L. and Garber, S. M., Surgical smokea review of the literature: Is this just a lot of hot air?, Surg Endosc, Vol 17, pp 979-987. 2003.
¹⁹ Garden, J M and O'Banion, K M and Shelnitz, L S and et al., Papillomavirus in the vapor of carbon dioxide laser-treated verrucae, Journal of American Medical Association, Vol 259, pp 1199-1202, 1988.
²⁰ Hill, D S and ONeill, J K and Powell, R J and Oliver, D W, Surgical smoke—a health hazard in the operating theatre: a study to quantify exposure and a survey of the use of smoke extractor systems in UK plastic surgery units, J Plast Reconstr Aesthet Surg., Vol 65, No 7, pp 911-916, 2012.
²¹ Semple, S and Ibrahim, A E and Apsley, A and Steiner, M and Turner, S, Using a new, low-cost air quality sensor to quantify second-hand smoke (SHS) levels in homes, Tob Control, Vol 2, pp 153-8, 2015.
²² Stadnytskyi V, Bax C E, Bax A, Anfinrud P. The airborne lifetime of small speech droplets and their potential importance in SARS-CoV-2 transmission. Proc Natl Acad Sci USA. 2020; 117(22):11875-7. Available from: https://doi.org/10.1073/pnas.2006874117
²³ [Garbey 2020a]
Garbey, M.: Joerger. G.; Futrr, S. A Systems Approach to Assess Transport and Diffusion of Hazardous Airborne Particles in a Large Surgical Suite: Potential Impacts on Viral Airborne Transmission, Int. J. Environ. Res. Public Health 2020, 17, 5404.
²⁴ [Garbey 2020b]
Marc Garbey, Guillaume Joerger, Shannon Furr, Vid Fikfak, A Model of Workflow in the Hospital During a Pandemic to Assist Management, medRxiv, 2020
²⁵ Gastroenterology Professional Society Guidance on Endoscopic Procedures During the COVID-19 Pandemic, 2020, https://vww.gastro.org/practiceguidance/practice-updates/covid-19/gastroenterology-professional-societyguidance-on-endoscopic-procedures-during-the-covid-19-pandemic
²⁶ Shahnaz Sultan, Joseph K. Lim, Osama Altayar, Perica Davitkov, Joseph D. Feuer stein, Shazia M. Siddique, Yngve Falck-Ytter, and Hashem B. El-Serag, on behalf of the American Gastroenterological Association AGA institute Rapid Recommendations for Gastrointestinal Procedures During the COVID-19 Pandemic Gastroenterology 2020, pp 120, in press.
²⁷ Garbey M. Joerger G. Furr S. Gastroenterology Procedures Generate Aerosols: An Air Quality Turnover Solution to Mitigate COVID-19's Propagation Risk. Int J Environ Res Public Health. 2020 Nov. 26; 17(23):8780. doi: 10.3390/ijerph17238780. PMID: 33256004; PMCID: PMC7731398.
²⁸ http://aqicn.org/calculator
²⁹ https://www.airnow.gov/aqi/aqi-calculator/
³⁰ Tove Hoffman and Karolina Nissen and Janina Krambrich and Bengt Rnnberg and Dario Akaberi and Mouna Esmaeilzadeh and Erik Salaneck and Johanna Lindahl and ke Lundkvist, Evaluation of a COVID-19 IgM and IgG rapid test; an efficient tool for assessment of past exposure to SARS-CoV-2. Infection Ecology & Epidemiology, Vol 10, No 1, 2020, Taylor & Francis.
³¹ Scohy A, Anantharajah A, Bodus M, Kabamba-Mukadi B. Verroken A, Rodriguez Villalobos H. Low performance of rapid antigen detection test as frontline testing for COVID-19 diagnosis. J Clin Virol. 2020; 129:104455. doi:10.1016/j.jcv.2020.104455
³² Franklin Dexter and Mohamed Elhakim and Randy W. Loftus and Melinda S. Seering and Richard H. Epstein Strategies for daily operating room management of ambulatory surgery centers following resolution of the acute phase of the COVID-19pandemic, Journal of Clinical Anesthesia. Vol 64, 2020, http://www.sciencedirect.com/science/article/pii/S0952818020306176
³³ Contributor Price Industries Engineer's HVAC Handbook A Comprehensive Guide to HVAC Fundamentals, 2011, Price Industries Limited.
³⁴ (Table 1) American Conference of Governmental Industrial Hygienists (ACGIH). HVAC components, functions and malfunctions (topic 8-4), In Industrial ventilation: a Manual of recommended practice, 24th ed. Cincinnati, OH: American Conference of Governmental Industrial Hygienists, Inc. 2001.
³⁵ https://www.cdc.gov/infectioncontrol/pdf/guidelines/environmental-guidelines-P.pdf
³⁶ Garbey M, Joerger G, Furr S. Gastroenterology Procedures Generate Aerosols: An Air Quality Turnover Solution to Mitigate COVID-19's Propagation Risk. Int J Environ Res Public Health. 2020; 17(23):8780. Published 2020 Nov. 26. doi:10.3390/ijerph17238780
³⁷ Industrial Ventilation: A Manual of Recommended Practice. 24th ed. The American Conference of Governmental Industrial Hygienists, Inc. (ACGIH); Cincinnati, OH. USA: 2001.
³⁸ Movement of airborne contaminants in a hospital isolation room. Eames I. Shoaib D, Klettner C A, Taban V J R Soc Interface. 2009 Dec. 6; 6 Suppl 6:S757-66.
³⁹ Liu, Y.; Song, Y.; Hu. X.; Yan. L.; Zhu, X. Review Awareness of surgical smoke hazards and enhancement of surgical smoke prevention among the gynecologists. J Cancer 2019, 10, 2788-2799.
⁴⁰ Romano, F.; Gusten, J.; De Antonellis, S.; Joppolo, C. M. Electrosurgical Smoke: Ultrafine Particle Measurements and Work Environment Quality in Different Operating Theatres. Int. J. Environ. Res. Public Health 2017, 14, 137.
⁴¹ Meeuwsen, F.; Guédon, A.; Arkenbout, E.; van der Elst, M.; Dankelman. J.; van den Dobbelsteen, J. The Art of Electrosurgery: Trainees and Experts. Surg. Innov. 2017, 24. 373-378.
⁴² Tang, J.; Nicolle, A.; Pantelic, J.; Klettner, C.; Su. R.; Kalliomaki, P.; Saarinen, P.; Koskela, H.; Reijula, K.; Mustakallio, P.; et al. Different types of door-opening motions as contributing factors to containment failures in hospital isolation rooms. PLoS ONE 2013, 8, e66663.
⁴³ Huang, Albert & Joerger, Guillaume & Salmon, Remi & Dunkin, Brian & Sherman, Vadim & Bass, Barbara & Garbey, Marc. (2016). A robust and non-obtrusive automatic event tracking system for operating room management to improve patient care. Surgical endoscopy. 30. 10.1007/s00464-015-4610-2.
⁴⁴ https://www.massgeneral.org/assets/MGH/pdf/news/coronavius/list-of-aerosol-generating-procedures.pdf.

1. A system for providing for risk mitigation of aerosolized particulates in a closed space, comprising:
an air quality sensor or plurality of sensors and monitoring device or plurality of monitoring devices;
said air quality sensor or sensors monitoring and quantifying aerosolized small particles in the range of 0.5 microns to 10 microns over time;
said air quality sensor monitoring and quantifying aerosolized small particles 2.5 microns or less from said 0.5 to 10 microns range over time;
a microcomputer connected to said air quality monitor and an onsite server for collection of acquired particle size data;
a server connected to said microcomputer and a storage database;
said database connected to said onsite sever;
a wired or wireless connection for connection of onsite server to a hosted webserver;
a computer or mobile application for receiving information related to air quality in a closed space; and
a graphic user interface (GUI) for communicating data to a user allowing user to monitor, track and assess a space's room state and ambient air quality and air quality turnover;
said GUI views is adjustable and configurable by the user.

2. The system of claim 1, wherein collected air quality data may be combined with additional space data including:
a computer vision sensor connected to any imaging device present in said space (ex. endoscope, surgical robot, microscope);
monitored space temperature, pressure, humidity and air velocity data;
aerosolized cleaning product data;
spatial patient, staff and equipment location data via BLE or RFID tags; and
door open and door closed conformations.

3. The system of claim 1, wherein said air quality sensor is capable of monitoring particle size in the diameter ranges:
microns, 10 microns or less, between 10 microns and 5 microns, 5 microns, 5 micron or less, 2.5 microns, 2.5 micron or less, 0.5 micron and above, between 0.5 micron and 2.5 microns, between 0.5 micron and 5 microns, between 0.5 micron and 10 microns, or a combination thereof.

4. The system of claim 3, wherein said air quality sensor collects data in real time, at predetermined intervals, times or a combination thereof.

5. The system of claim 4, wherein data is collected for small molecules in the 0.5 to 2.5 micron-diameter range, expressed as 2.5 microns and below, which is then converted to an absolute designation correlating to an Air Quality Index (AQI).

6. The system of claim 5 wherein an AQI of 50 or less designates “excellent” air quality which relates to decreased risk of exposure to aerosolized vectors which may transmit disease-causing bacteria, viruses and fungi.

7. The system of claim 6 wherein said air quality sensor data, once collected and transmitted to a user, is displayed wherein user is informed of one or a combination of the following:
air quality
number of monitored spaces;
timelines for each space;
activity within said each space including space preparation, occupation of space, presence of space activity, confirmation of activity start, ongoing activity or end of activity;
accumulated exposure to hazardous particulate quality in real time or over time; or
a combination thereof.

8. The system of claim 7, wherein said activity space, being amendable to closure, comprises:
(a) a space within a medical facility which may be a clinic, surgical suite, operating room, transport area, receiving area, or medical office space;
(b) in a space generally available to the public, or
(c) a combination thereof.

9. The system of claim 8 wherein said activity is a surgical procedure or surgical activity.

10. The system of claim 9 wherein said surgical procedure or surgical activity is a general surgery, such as appendectomy or cholecystectomy or a gastroenterology (GI) procedure such as colonoscopy, esophagogastroduodenoscopy (EGD), or Endoscopic Retrograde Cholangiopancreatography (ERCP), or a combination thereof.

11. The system of claim 10 wherein data monitored, collected and analyzed is utilized to test Heating, Ventilation and Air Conditioning (HVAC) systems, monitor HVAC systems, better utilize and support HVAC systems in an effort to safely protect patients, safeguard surgical staff, increase surgical throughput, increase surgical flowrates and improve surgical suite turnover times through air quality monitoring.

12. The system of claim 8, wherein data monitored, collected and analyzed is utilized to mitigate pathogen exposure in a space for surgical activity, generally available to the public, or a combination thereof.

13. An air quality sensor unit or plurality of sensor units for the detection and monitoring of aerosolized particles utilizable in claim 1 which is modular in construction with 3 compartments, comprising:
two exterior residing compartments and one centralized compartment;
said one of the two exterior compartments housing a power source connection, a microcomputer and a means for communication;
said communication means being a wireless antenna, a wired antenna, or a combination thereof;
said other of two compartments housing an ancillary infrared, ultrasonic sensor for motion, an ultrasonic sensor for assessing door confirmation, or a combination thereof;
said centralized compartment housing one to a plurality of sensors

14. The air quality sensor unit for the detection and monitoring of aerosolized particles of claim 13, wherein said centralized compartment sensors may be a gas sensor for volatile organic compounds, an optical laser particle count sensor, multiple and redundant functionally equivalent sensors, or a combination thereof.

15. The air quality sensor unit for the detection and monitoring of aerosolized particles of claim 13, wherein said ancillary sensors may be an ultrasonic sensor for door monitoring, infrared sensor for motion detection, a light sensor, a noise sensor, or a combination thereof.

16. The air quality sensor unit for the detection and monitoring of aerosolized particles of claim 13, wherein said unit or units may be equipped with an antenna to capture Bluetooth low energy (BLE) or Radio-frequency identification (RFID) signals from patients, staff, doors and equipment.
